(19) United States
(12) Patent Application Publication
(10) Pub. No.: US 2017/0000359 A1
(43) Pub. Date: Jan. 5, 2017

(54) COMPREHENSIVE BODY VITAL SIGN MONITORING SYSTEM

(71) Applicant: Cloud Ds, Inc., a corporation of Delaware

(72) Inventors: Sandeep S. Kohli, Oakville (CA); Robert Kaul, Brooklyn, NY (US); Anthony Kaul, Sidney (CA); Sara Ross-Howe, Campbellville (CA); Justin Pedro, Waterloo (CA)

(21) Appl. No.: 15/268,556

(22) Filed: Sep. 17, 2016

Related U.S. Application Data
(63) Continuation-in-part of application No. 15/060,514, filed on Mar. 3, 2016.
Application No. 15/060,514 is a continuation of application No. 14/186,151, filed on Feb. 21, 2014.
(60) Provisional application No. 62/138,377, filed on Mar. 25, 2015.
(60) Provisional application No. 61/767,839, filed on Feb. 22, 2013.

(51) Int. Cl. A61B 5/0205; A61B 5/0408; A61B 5/145; A61B 5/00

(52) U.S. Cl. CPC A61B 5/02055; A61B 5/6823; A61B 5/04085; A61B 5/14542; A61B 5/0022; A61B 5/0006; A61B 5/742; A61B 5/6815; A61B 5/6826; A61B 5/6824; A61B 5/4818; A61B 5/7475; A61B 5/14532; A61B 5/4833; A61B 5/021

(57) ABSTRACT

Ambulatory patient wearable vital sign monitoring system, often comprising a plurality of ambulatory patient wearable ECG sensors, pulse oximeters, and oscillometric blood pressure monitors, configured to transmit sensor data to a wearable computerized device such as a smartphone. The smartphone will typically be configured to obtain the patient's health history and other health status information, often by querying the patient. The device will use this health history and health status information to select vital sign operating times and sensor data acquisition parameters. The system may also use the history, health status information and the sensor data to return diagnostic information pertaining to the patient's history the patient and optionally other devices or caregivers. In some embodiments, the system may also report on medication adherence as well. Examples of multi-sensor neck worn devices, three-sensor hand carried devices, and purely ear worn devices (e.g. for sleep apnea) are presented.

### CROSS REFERENCE TO RELATED APPLICATIONS

This application is a continuation in part of U.S. patent application Ser. No. 15/060,514, “SYSTEMS AND METHODS FOR MONITORING PATIENT MEDICATION ADHERENCE”, filed Mar. 3, 2016; Application Ser. No. 15/060,514 claimed the priority benefit of U.S. provisional application 62/138,377, “COMPREHENSIVE BODY VITAL SIGN MONITORING SYSTEM WITH NECK AND EAR MOUNTED DEVICE” filed Mar. 25, 2015; This application is also a continuation in part of U.S. patent application Ser. No. 14/186,151, “SIMULTANIOUS MULTI-PARAMETER PHYSIOLOGICAL MONITORING DEVICE WITH LOCAL AND REMOTE ANALYTICAL CAPABILITY”, filed Feb. 21, 2014; Ser. No. 14/186,151 claimed the priority benefit of U.S. provisional application 61/767,839, filed Feb. 22, 2013; the entire contents of all of these applications are incorporated herein by reference.

### BACKGROUND OF THE INVENTION

Field of the Invention

This invention is in the field of medical diagnostics.

Description of the Related Art

As medicine has advanced, and as the art of electronics and medical instrumentation has advanced, there has been increased interest in recent years in pushing sophisticated medical diagnostics tests out of the hospital and clinic and closer to the patient's everyday life and environment.

For example, home blood pressure tests, and home blood glucose tests, have transformed the standard of care for hypertension and diabetes, because these tests enable patients to obtain much greater control over their medical conditions. Other types of cardiac function tests, such as Holter monitors, allow the function of the patient's heart to be assessed in the patient's natural environment, rather than in a doctor's office or clinic. This enables a much broader range of cardiac issues to be diagnosed.

Despite these advances, the art of pushing medical diagnostics out of the clinic and into the patient's daily life is still at a comparatively early stage, and here further advances in this art would be highly useful.

### BRIEF SUMMARY OF THE INVENTION

In some embodiments, the invention may comprise an ambulatory patient wearable vital sign monitoring system or method. The system and method will generally comprise a plurality of ambulatory patient wearable sensors, each wearable sensor comprising at least one ECG sensor, pulse oximeter, and oscillometric blood pressure monitor. Each wearable sensor will typically be further configured to transmit sensor data to an ambulatory patient wearable computerized device.

This ambulatory patient wearable computerized device will generally comprise at least one processor (e.g. a computer processor, microprocessor, and the like), and at least one user interface. This ambulatory patient wearable computerized device will typically be configured to use this at least one user interface to query an ambulatory patient with various questions regarding a health history of the ambulatory patient, and receive health status information pertaining to the ambulatory patient's health history.

The ambulatory patient wearable computerized device will typically be further configured to use this health status information to select time duration and sensor data acquisition parameters used by the vital sign monitoring system. Additionally or alternatively, the ambulatory patient wearable computerized device will typically be configured to use this health status information and the sensor data to return diagnostic information pertaining to the patient's health (e.g. health status, medication status) to at least the ambulatory patient, and possibly to additional computerized devices (e.g. remote internet devices or databases), and/or caregivers (either local or remote) as well.

The configuration of the various ambulatory patient wearable sensors can vary. In some embodiments, at least some of the sensors may be provided in the form of a neck mounted devices. In other embodiments, each sensor may be an individually worn sensor, and a plurality of such individually worn sensors may then interact with the patient wearable computerized device by either wireless links (e.g. Bluetooth™, WiFi), wired links, optical (e.g. infrared links) or other methods. In some of the embodiments, at least some of the sensors may be hand carried sensors. Although in this disclosure, many of the specific figures and examples show a neck mounted configuration with an optional ear device, and other optional sensor components, these examples are not intended to be limiting.

Further, although it is contemplated that at a minimum, the system and method will use a plurality of sensors comprising at least an ECG sensor, pulse oximeter, and oscillometric blood pressure monitor, it will often be desirable to employ a larger number of sensors. In some embodiments, this larger number of sensors may include temperature sensors, blood glucose monitors, breathing sensors, and the like. In general, more sensors tend to be desirable as this can provide a more comprehensive overview of the patient's health status, but of course the expenses and calibration problems associated with adding a large number of additional sensors are also a consideration.

In some embodiments, the invention may comprise a comprehensive body vital monitoring sign system that has, as at least one component, a wearable electronic neck and ear device with both various onboard vital sign sensors, such as electrocardiogram (ECG) electrode sensors, peripheral capillary oxygen saturation (SPO2) sensors, photoplethysmogram (PPG) sensors, thermopile sensors (infrared sensors configured for temperature measurement), as well as additional user motion and environmental sensors such as accelerometers, gyroscopes, global positioning system (GPS) sensors, and the like. The neck component of this wearable neck device may be formed from various rigid and soft components, and may also employ multiple hot-swappable batteries for uninterrupted, continuous operation.

The wearable ear device may, in some embodiments, comprise a blood oxygen sensor and a body core temperature monitor. For example, the wearable ear device may also function to produce photoplethysmography (PPG) data. In these embodiments, it may be useful to configure the wearable ear device so that it performs photoplethysmography determinations on various ear regions such as the concha and anti-tragus Area of the ear. Additionally, the ear device may also be configured with an infrared sensor or other type sensor positioned and calibrated so as to obtain body core temperature measurements from the ear canal.

In some embodiments, the system may also comprise a handheld “wand” with a built in light source, video camera, and lens system designed to fit into the ear, as well as other components so that the wand device can operate as a combination wireless Otoscope and Spirometer. Here this Otoscope/Spirometer may transmit results to a local computerized device, such as a smartphone, where software (such as a user app) and associated user interface can be used to both help operate and help interpret data obtained from the wand device.

In some embodiments, data, such as video from the wand device's video camera system, may be interpreted on this local computerized device using various types of software. This software can include various methods of user (or computer) based medical assessment using comparative analysis of positively and negatively diagnosed standards.

In some embodiments, the system may also comprise a wrist mounted blood pressure cuff, configured to obtain blood pressure data from a user, and report this data, often using a wireless transceiver, to a local computerized device.

In other embodiments, data from the invention's electronic neck and ear device, and other peripherals, may be combined with various other types of in-vitro diagnostics as well. Such in-vitro diagnostics may, for example, operate from small drops of blood obtained from the user by various lancing methods, however other methods may also be used.

In this regard, in some embodiments, simplified methods of in-vitro diagnostics, such as methods employing assay cartridges with integrated lancing, blood collection, activator storage, and sample dispensing may be useful.

The system will typically further employ a unique software based integrated sensor control and medical diagnosis system, configured to operate on common handheld computerized devices such as smartphones, that is designed to allow the user to comprehensively integrate sensor data from many different sensors, solicit further medical information from the user, and report at least tentative medical diagnosis and/or body state information based on these various results.

### BRIEF DESCRIPTION OF THE DRAWINGS

FIGS. 1A to 1G show various views of the invention's combination neck worn physiological and environmental monitoring system with optional ear attachment. FIG. 1A shows this system from the front (patient not shown).

FIG. 1B shows this system from the back.

FIG. 1C shows this system from the side.

FIG. 1D shows a transparent view of the system showing some of the details of the internal circuitry.

FIG. 1E shows a transparent view of the system's combination battery holder/electrode unit, and a transparent view of the system's optional ear device.

FIG. 1F shows an example of how the system fits around the neck of a user.

FIG. 1G shows an example of how the optional earpiece device can fit into the user's ear.

FIG. 2A shows an exploded view of the invention's optional earpiece attachment from the front.

FIG. 2B shows an exploded view of the invention's optional earpiece attachment from the back.

FIG. 2C shows an exploded view of the internal circuitry of the optional earpiece attachment, along with more details the earpiece housing.

FIG. 3A (and FIGS. 3B to 3V show) software flow charts showing some of the various medical diagnostic algorithms that the system may implement, usually by way of a local computerized device such as a smartphone. FIG. 3A shows an overview of the process.

FIG. 3B shows more detail of the invention's diagnostic flows and tester scenarios.

FIG. 3C shows more detail of the invention's diagnostic continuum that accesses various diagnostic factors and questions and assigns and ranks the results according to how likely the results are symptomatic of an underlying disease.

FIG. 3D shows a detail of the inventions' diagnostic flow question and answer architecture.

FIG. 3E shows a detail of the invention's diagnostic flow recommendation's architecture (e.g. diagnostic recommendations and advice).

FIG. 3F shows a detail of some of the invention's diagnostic flow recommendation rules.

FIG. 3G shows a detail of one of the invention's diagnostic flow outcome probability calculation algorithms.

FIG. 3H shows a partial example of a diagnostic flow for pneumonia (1 of 2).

FIG. 3I shows a partial example of a diagnostic flow for pneumonia (2 of 2).

FIG. 3J shows a partial example of a diagnostic flow for acute hemorrhagic stroke.

FIG. 3K shows a partial example of a diagnostic flow for acute otitis media.

FIG. 3L shows a partial example of a diagnostic flow for hepatitis A.

FIG. 3M shows a partial example of a diagnostic flow for urinary tract infection.

FIG. 3N shows a partial example of a diagnostic flow for tuberculosis.

FIG. 3O shows a partial example of a diagnostic flow for mononucleosis.

FIG. 3P shows a partial example of a diagnostic flow for chronic obstructive pulmonary disease (COPD).

FIG. 3Q shows a partial example of a diagnostic flow for obstructive sleep apnea.

FIG. 3R shows a partial example of a diagnostic flow for hypertension.

FIG. 3S shows a partial example of a diagnostic flow for atrial fibrillation.

FIG. 3T shows a partial example of a diagnostic flow for diabetes.

FIG. 3U shows a partial example of a diagnostic flow for anemia.

FIG. 3V shows a partial example of a diagnostic flow for leukocytosis.

FIG. 3W shows a partial example of a diagnostic flow for high LDL cholesterol.

FIG. 4A (and FIGS. 4B to 4AD) shows an example of the user interface of the software that controls the various system components, and how this user interface can work with the various medical diagnostic algorithms previously shown in FIGS. 3A to 3W, as well as the various vital sign sensors shown in FIGS. 1A to 1G, 2A to 2C, and FIG. 5, to monitor various aspect of the user's health and physical fitness. 4A shows an introductory user interface.

FIG. 4B shows an example of a device setup user interface.

FIG. 4C shows an example of a start/profile screen user interface.

FIG. 4D shows an example of a first part of a “diagnose me” vital sign user interface.

FIG. 4E shows an example of a second part of a “diagnose me” vital sign user interface.

FIG. 4F shows an example of the user interface for some of the tier-1 symptoms that the system will help diagnose.

FIG. 4G shows an example of the user interface for some of the tier-1 pain symptoms.

FIG. 4H shows an example of the user interface for some of the body diagrams used by the system diagnosis methods.

FIG. 4I shows an example of the user interface for some of the Tier 2 and Tier 3 questions that the system diagnosis methods may also use.

FIG. 4J shows an example of the user interface for various user medical questions.

FIG. 4K shows an example of the user interface to diagnose otitis media (1 of 2).

FIG. 4L shows an example of the user interface to diagnose otitis media (2 of 2).

FIG. 4M shows an example of the user interface to use the system spirometer to help diagnose COPD (1 of 2).

FIG. 4N shows an example of the user interface to use the system spirometer to diagnose COPD (2 of 2).

FIG. 4O shows an example of the user interface to use an ultrasonic scanner (sonicvision) to diagnose stroke (1 of 2).

FIG. 4P shows an example of the user interface to use an ultrasonic scanner (sonicvision) to diagnose stroke (2 of 2).

FIG. 4Q shows an example of the user interface start page for various blood analysis tests.

FIG. 4R shows an example of the user interface start page for a blood cholesterol test.

FIG. 4S shows an example of the user interface results pages for blood cholesterol and blood glucose tests.

FIG. 4T shows an example of the user interface results page for a white blood cell count test and a hepatitis A test.

FIG. 4U shows an example of the user interface result pages for various types of diseases.

FIG. 4V shows an example of the user interface used to configure the system for an extended 72 hour series of vital sign monitoring.

FIG. 4W shows an example of the user interface used to show all actively monitored vital signs at once.

FIG. 4X shows an example of the user interface used to monitor heart rate and present a heart rate history.

FIG. 4Y shows an example of the user interface used to monitor respiration rate and present a respiration rate history.

FIG. 4Z shows an example of the user interface used to monitor blood oxygen saturation and present a blood oxygen saturation rate history.

FIG. 4AA shows an example of the user interface used to monitor blood pressure and present a blood pressure history.

FIG. 4AB shows an example of the user interface used to monitor body temperature and present a body temperature history.

FIG. 4AC shows an example of the user interface used to present various system alert messages to the user.

FIG. 4AD shows an example of the user interface used to present final diagnostic results to the user.

FIG. 5 shows an example of the in-vitro diagnostics meter component of the system (1500), which can monitor various analytes in the user's blood and urine (e.g. cholesterol, glucose, etc.) via various chemical analysis methods through port (1501). This in-vitro diagnostics meter unit (1500) may additionally be configured to perform other tasks as well, such as recharging batteries (1502) for the neck worn device (here 1503 can be a battery charge indicator), acting as a smartphone (1504) charging station, and acting as a recharge station for other diagnostic system devices as well, such as blood pressure cuffs (1506), cameras, spirometers, ultrasonic scanners, and the like (1508).

FIG. 6 shows a simplified electrical schematic of a multi-parameter monitoring device produced according to the teaching of parent application Ser. No. 14/186,151, the entire contents of which are incorporated herein by reference.

FIG. 7 shows a flow chart showing of some of the various steps that may be carried out the system's processor in order to determine if the various physiological measurements taken by the device's various sensors are showing that the patient is likely following his or her assigned medication schedule, or not, according to the teaching of parent application Ser. No. 15/060,514, the entire contents of which are incorporated herein by reference.

FIG. 8 shows that in some embodiments, the device may also communicate with an external server.

### DETAILED DESCRIPTION OF THE INVENTION

As previously discussed, in some embodiments, the invention may comprise an ambulatory patient wearable vital sign monitoring system or method. The system will generally comprise a plurality of ambulatory patient wearable sensors, each wearable sensor comprising at least one ECG sensor, pulse oximeter, and oscillometric blood pressure monitor. Each wearable sensor will typically be further configured to transmit sensor data to an ambulatory patient wearable computerized device.

This ambulatory patient wearable computerized device will generally comprise at least one processor (e.g. a computer processor, microprocessor, and the like), and at least one user interface. This ambulatory patient wearable computerized device will typically be configured to use this at least one user interface to query an ambulatory patient with a plurality of queries regarding a health history of the ambulatory patient, and receive health status information pertaining to the ambulatory patient's health history.

The ambulatory patient wearable computerized device will typically be further configured to use this health status information to select a time duration and sensor data acquisition parameters of the vital sign monitoring system. Additionally or alternatively, the ambulatory patient wearable computerized device will typically be configured to use this health status information and the sensor data to return diagnostic information pertaining to the health history to at least the ambulatory patient, and possibly additional computerized devices or caregivers as well.

These wearable sensors may be configured into different types of devices, including neck worn devices (discussed in more detail shortly), handheld devices (discussed previously in parent application Ser. No. 14/186,151, the entire contents of which are incorporated herein by reference), and even individually worn devices (e.g. a plurality of different patient worn sensors, all communicating with at least one common processor using any of wired or wireless links).

Although often the diagnostic information provided by the device will be used to help monitor the patient for the presence or absence or status of various disease states, in some embodiments, the diagnostic information may further pertain to the medication status of the patient. Here, the methods previously discussed in parent patent application Ser. No. 15/060,514, the entire contents of which are incorporated herein by reference, may be used.

As previously discussed, in one embodiment the invention may have a neck worn or mounted physiological and environmental monitoring components, with an optional ear attachment, which may also have some additional physiological sensors.

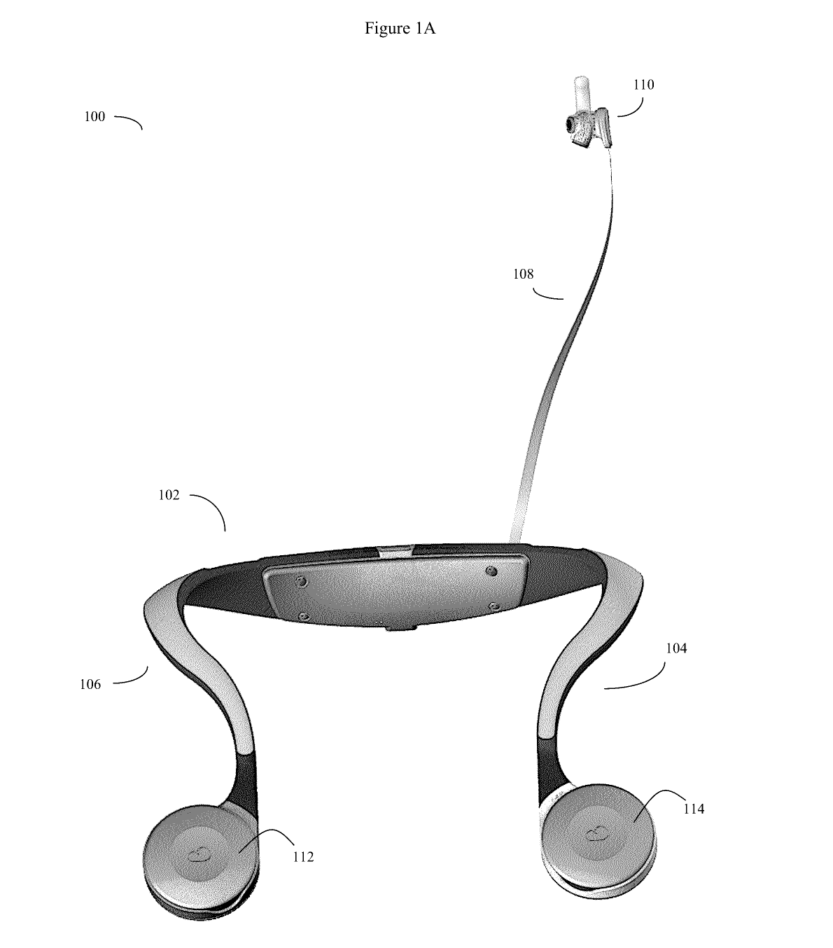

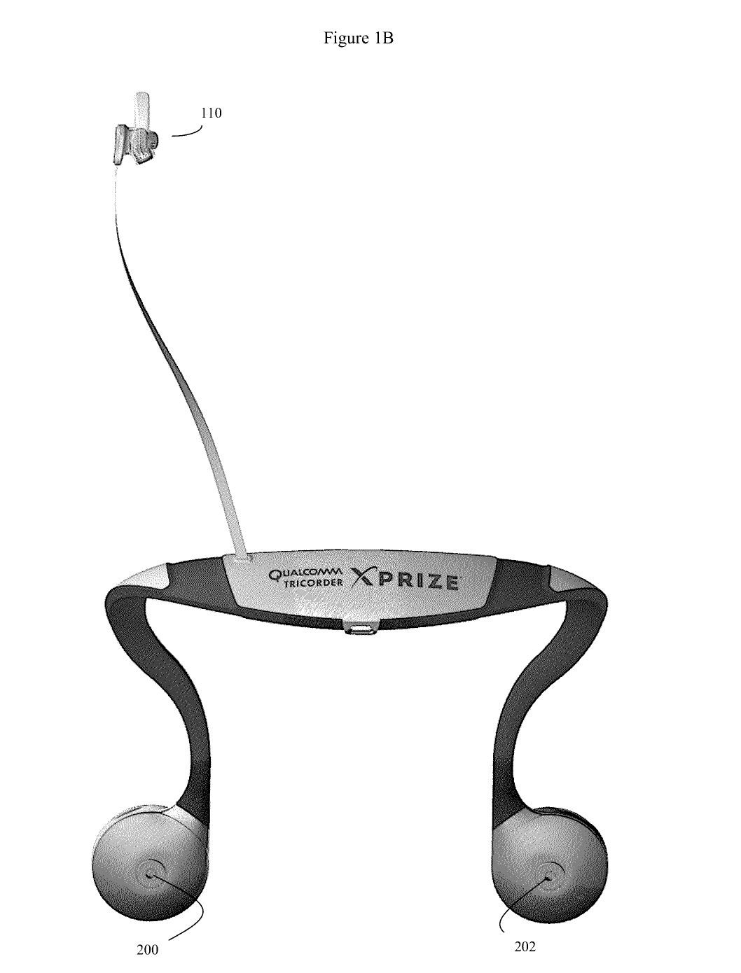

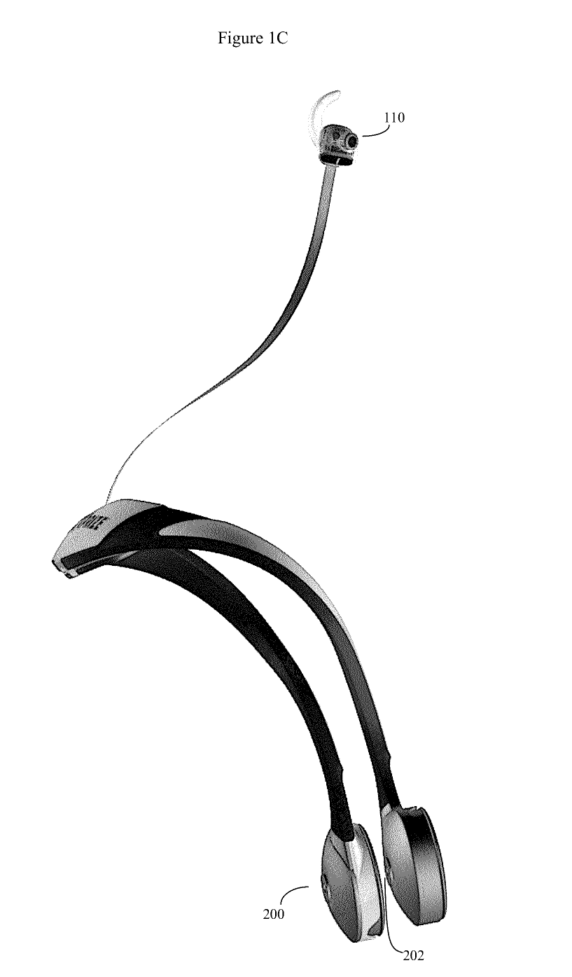

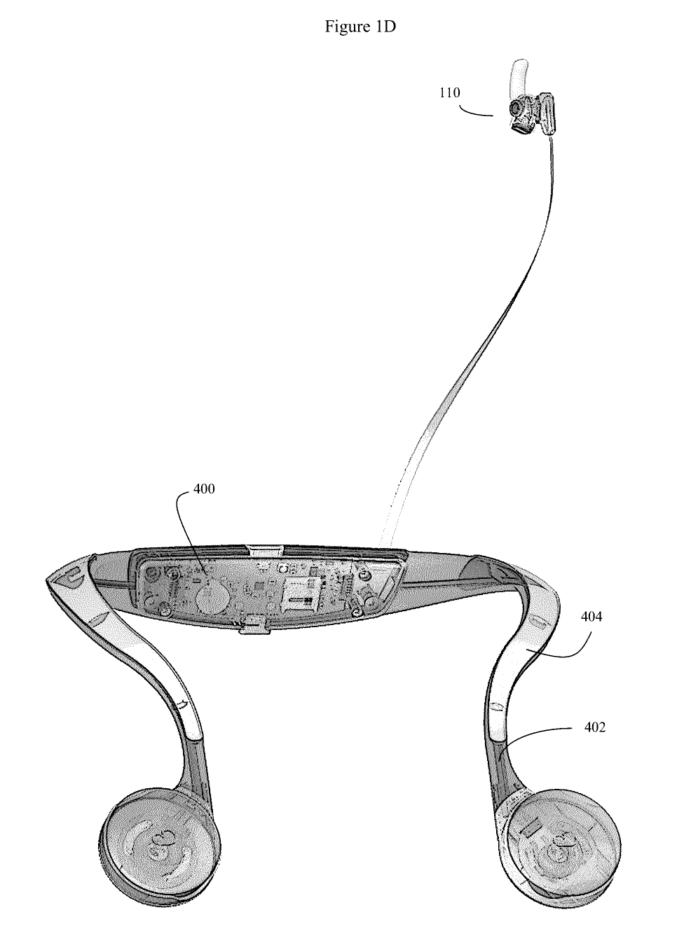

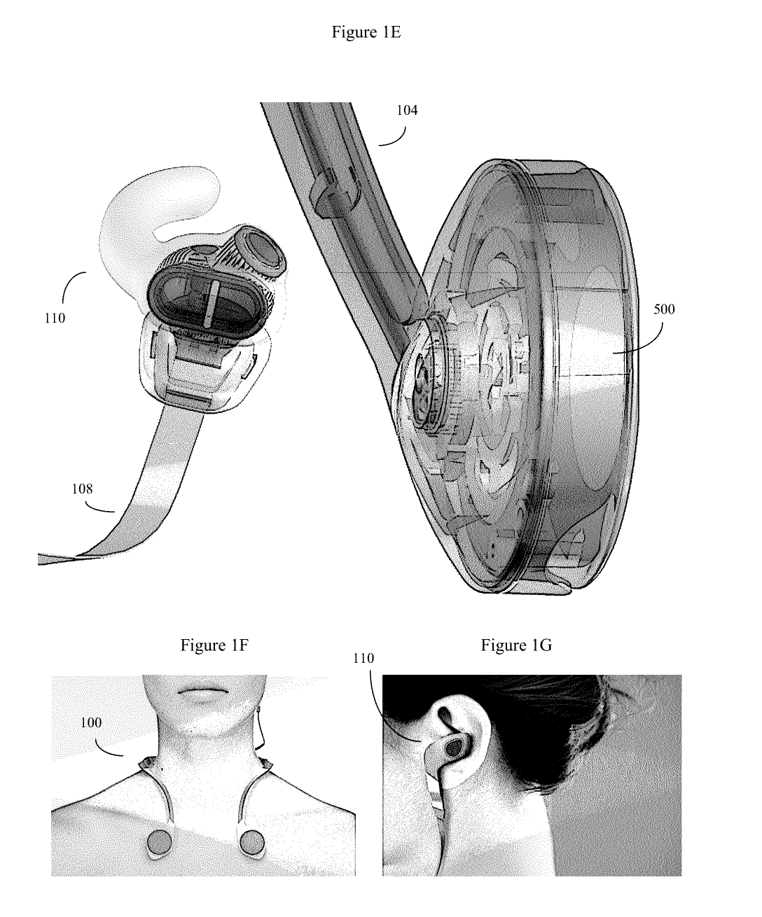

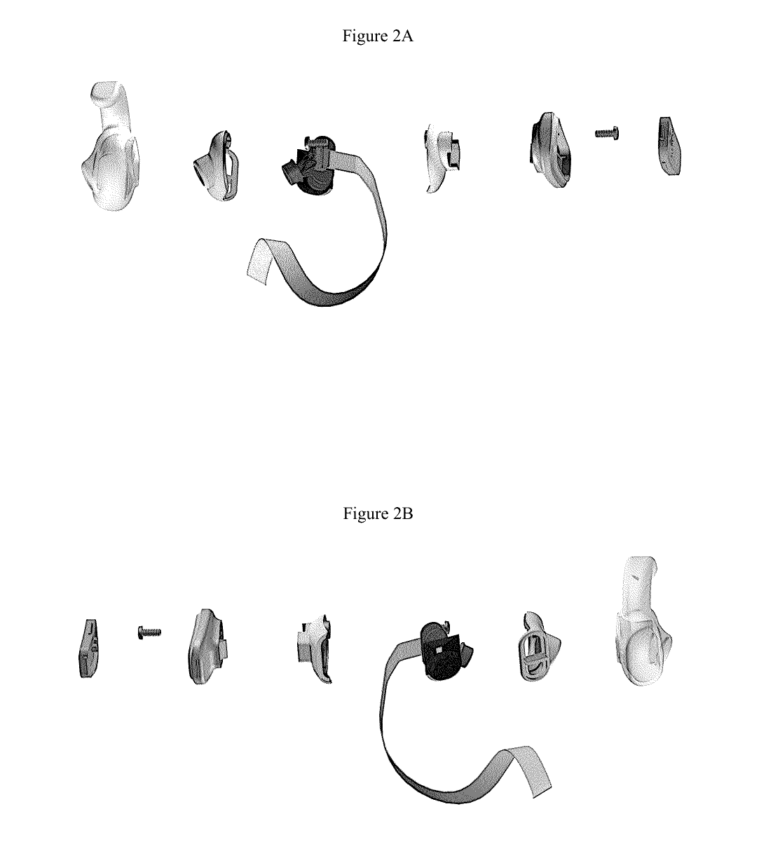

FIGS. 1A-1G show various views of a neck worn or mounted monitoring device that may be employed in this embodiment.

In this embodiment, the overall design of this neck worn device (100) may use an open front collar (102) which is easy to don, and whose form is comfortable to wear for extended periods. The device may typically have various physiological and environmental sensors.

These may comprise multiple onboard vital sign sensors, such as: ECG, SPO₂ (PPG), Thermopile sensors, as well as various environmental sensors such as accelerometers, gyroscopes, and GPS sensors.

The front of this device may have two adjustable arms (104, 106) which extend down towards the user's chest. At the bottom of these arms, on the side which faces the user's skin, a snap type fitting may be positioned to accept ECG electrodes, such as wet (sticky) ECG electrodes (200, 202). In this embodiment ECG data may be captured below the patient's clavicle, using two leads symmetric about the sternum which approximate the V1 (4^{th} intercostal space, right of the user's sternum) and V2 (4^{th} intercostal space, left of the user's sternum) leads of traditional ECG equipment.

As shown in FIGS. 1F and 1G, this neck mounted device may be worn around the user's neck and shoulders, and in some embodiments may also have an appendage (e.g. cable 108) going up the patient's ear(s) with an optional ear attachment device (110).

Thus in this embodiment, the neck mounted device (110) may contain at least an ECG sensor, batteries, an optional computer processor (see FIG. 1D, 400), memory, and circuitry to drive both the ECG sensors and other sensors. The optional earpiece will often also have PPG and Thermopile sensors, which may, for example, be driven by the electrical circuitry and power supplied by the neck mounted device through a cable (108), or by other methods such as internal earpiece batteries.

Thus in this embodiment, the system may comprise at least one ECG sensor configured as a chest wearable ECG sensor. Further, the pulse oximeter may be either an ear wearable or a finger wearable pulse oximeter. The oscillometric blood pressure monitor may be a wrist wearable oscillometric blood pressure monitor.

In some embodiments, this at least one ECG sensor may comprise a plurality of ECG sensors both attached to, and in electrical communication with, a neck mounted flexible harness.

Further, both the at least one pulse oximeter and the ambulatory patient wearable computerized device may optionally both be attached to, and in electrical communication with, the neck mounted flexible harness.

Alternatively, or additionally, as previously discussed, any of the one or more ECG sensors, pulse oximeters, and oscillometric blood pressure monitors may be configured to transmit data to the ambulatory patient wearable computerized device using a wireless link. The neck mounted device configuration is optional, and in some cases, as previously discussed, the various sensors may be mounted independently on the patient.

Returning to the neck mounted example, in some embodiments, the neck mounted device electronics may also include additional sensors, such as additional environmental sensors (e.g. accelerometers and/or gyroscopes to monitor user movement), GPS sensors to monitor the user's location, and the like. The neck mounted device may also include various methods to interface with local computerized devices such as smartphones, often by way of various short range wireless transceivers such as Bluetooth transceivers. The additional sensors can augment the vital sign sensors to provide vital sign information tied to location, activity, and activity duration.

In some embodiments, the rear of this neck device may house the main electronics assembly, batteries, positional sensors (Accelerometer, Gyroscope, and GPS), onboard computer, and wireless modules (See FIG. 1D, 400).

In some embodiments, the main electronics may utilize electronics such as the Intel® Edison Development Platform. This development board comprises Intel® Edison Board for Arduino, and supports the Arduino Sketch, and Linux (e.g. Yocto Linux v1.6) operating systems, and provides Wi-Fi, and Bluetooth wireless transceivers. The development board I/O includes 20 digital input/output pins (including 4 pins as PWM outputs), 6 analog inputs, 1 UART (Rx/Tx), 1 I2C interface, 1 ICSP 6-pin header (SPI interface), and any of a micro USB device connector or via a mechanical switch, a dedicated standard size USB host Type-A connector. The board's Micro USB device is connected to the board's UART. The board processor is based on a 22 nm Intel® SoC that includes a dual-core, dual-threaded Intel® Atom™ CPU running at 500 MHz and a 32-bit Intel® Quark™ microcontroller running at 100 MHz. The board also contains 1 GB RAM, and 4 GB Flash memory.

In some embodiments, the main electronics may be further supplemented by additional circuitry such as an accelerometer, a power management system, connections for the system's ECG, and wearable earpiece sensors (e.g. core body temperature & PPG sensors), as well as a non-user-accessible, backup battery to ensure continuous operation if not hot swap batteries are present. This daughterboard may be further supplemented by additional Gyroscopic or other local motion sensors, GPS receivers, even cellular LTE transceiver chips for added functionality.

From the rear enclosure, cables (108) exit the housing, to a separate device which fits into the user's ear. Within this ear device (110), sensors such as a photoplethysmography (PPG) sensor and thermopile (e.g. infrared sensor for core body measurements) may be housed.

Human ears have the advantage of being highly vascular, which ensures accuracy for blood oximetry measurement. Of the two most common areas to obtain SPO₂, one is the earlobe. Similarly, body core temperature can be accurately measured in the ear canal. For the former, a PPG sensor may be used to obtain Blood Oxygen Saturation (SPO₂) when installed into the ear concha. The later body core temperature measurement may be obtained by a thermopile device just outside the ear canal and whose sensor is aimed towards the inside of the ear canal.

These three sensors as described above can be used both independently, and in combination, to derive useful vital signs (e.g. Respiration Rate via SPO₂, and Blood Pressure via Pulse Transit Time (PTT)). When combined with the position sensors, this device can be used to generate vital sign data that could be compared to a user's distance exercised, steps taken, speed achieved, weight repetitions, and other activities.

The advantages of this approach is that the system obtains vital signs by making measurements in the same parts of the patient's body that are normally also measured by standard clinical grade medical equipment. This simplifies the signal interpretation process. Additional advantages of this approach are that by combining various vital signs (e.g. 5 different vital signs) along with additional sensors such as accelerometers, other movement sensors, GPS sensors and the like, the system can produce unique insights that correlate a user's activity level with the user body's physiological response.

Construction of the Wearable Electronic Neck Device

Any wearable device (100) worn around the neck for extended periods should both be comfortable, and also accommodate a broad spectrum of adult body sizes and shapes. This is especially true for devices meant to monitor an aging population, or to provide a physician with continuous vital sign capture throughout the day.

In some embodiments, to be both comfortable and accommodate a broad spectrum of body sizes, the wearable neck device may be constructed using, at least in part, soft, thermoplastic elastomers combined with posable (e.g. bendable to assume and hold a particular configuration) armatures along the length of the neck device. To help ensure that the neck device is easy to adjust quickly, rigid areas may be defined that to some extent restrict adjustment to two areas on the left and right sides.

Although the neck device may be produced in multiple sizes, it will often be useful to produce the neck device in a “one size fits all” configuration that is intended to fit most of the adult users. The device will ideally be constructed to be comfortable over extended periods, and also accommodate a large spectrum of the adult population (ideally 5th-95th percentile). To accomplish these goals, a soft durometer, elastomeric thermoplastic (TPE), ideally chosen for non-allergenic properties, may be used at least in regions where the neck device contacts the user's body. This soft durometer TPE material may form or cover much of the neck device as it wraps around the user's neck and down the user's chest.

A transparent view of the wearable neck device, showing some of its various internal and external components, is shown in FIG. 1D. In some embodiments, the wearable neck device may be formed, at least in part, by overmoulding one or more posable armature wires (402) with the TPE material, thus encasing the armature wire(s) with TPE. The armature wire(s) thus act as an internal skeleton for the wearable neck device. The armature wire or wires thus allow the user to bend the neck device, and have the neck device hold its bended shape afterwards, and repeat this bending and adjustment process as many times as needed.

The advantage of this approach is that by allowing for adjustment that is not predefined, different sized users can bend the neck device to a comfortable form that accommodates their unique body size and shape, while still allowing the neck device to maintain a configuration that positions the vital sign sensors, such as the ECG electrode leads) in required areas of the user's body. Here for example, the ECG electrode leads can still be located below the user's clavicle, in a preferred location that is symmetrical about the user's sternum).

Note that the neck device need not be a necklace (although in some embodiments, necklace configurations may be employed as desired). That is, there need not be any direct connection between the bottom mounted V1 and V2 electrodes (shown by their contacts (200, 202) that pass over the front of the user's chest.

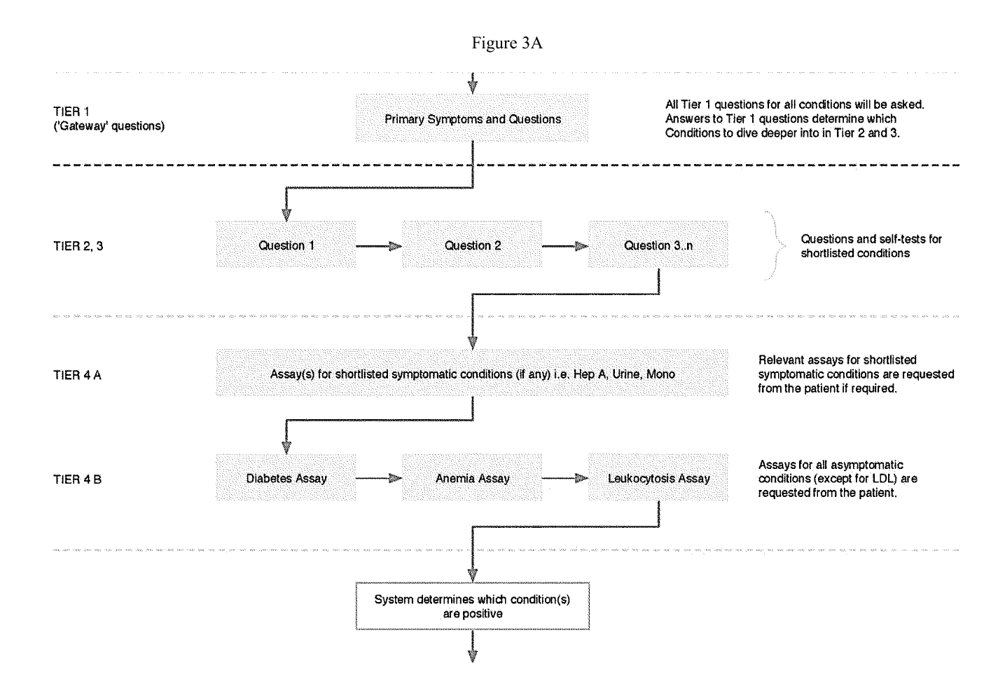

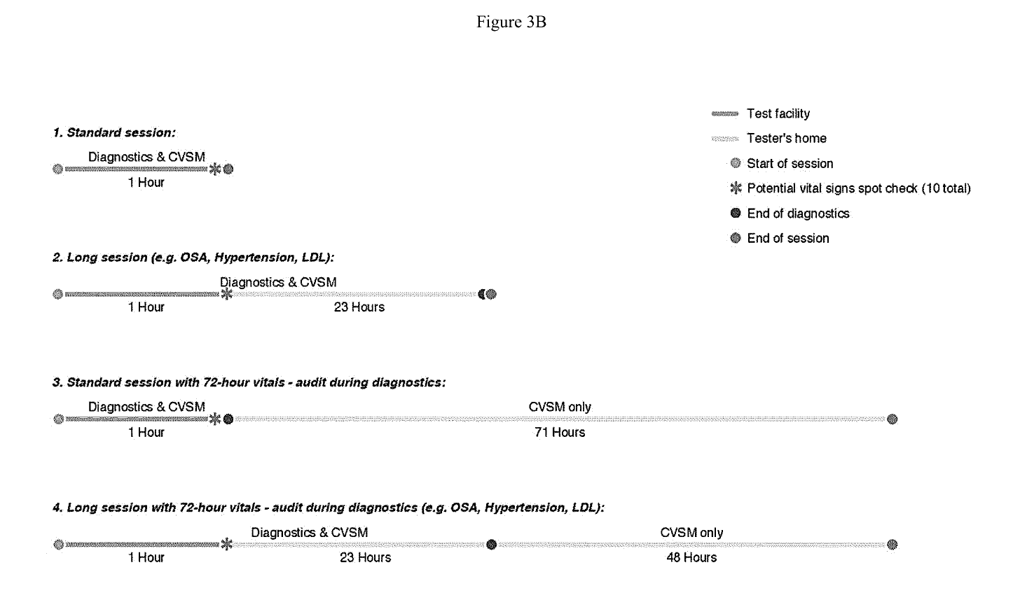

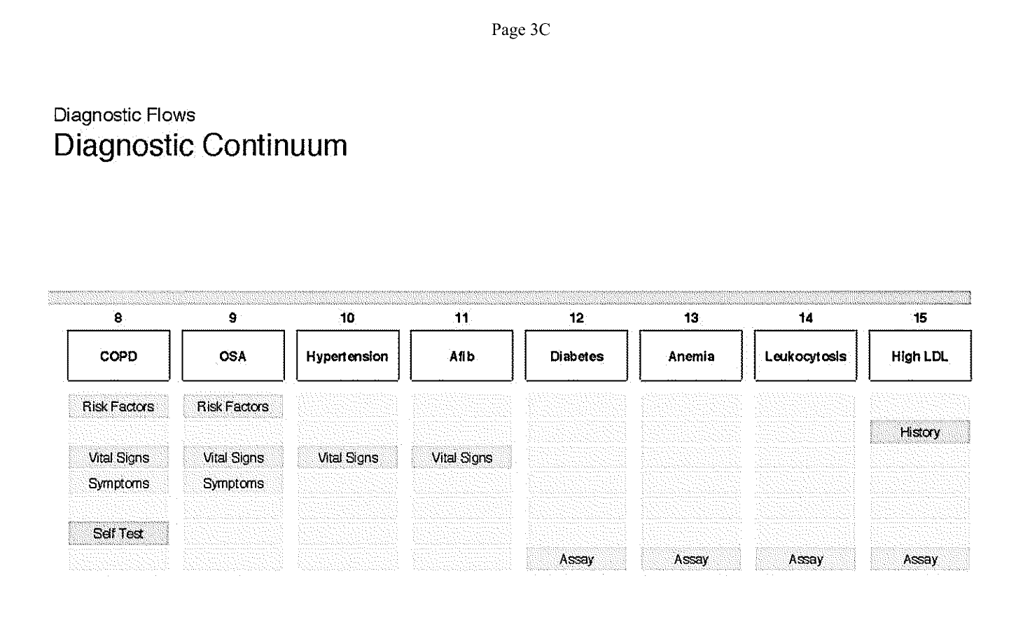

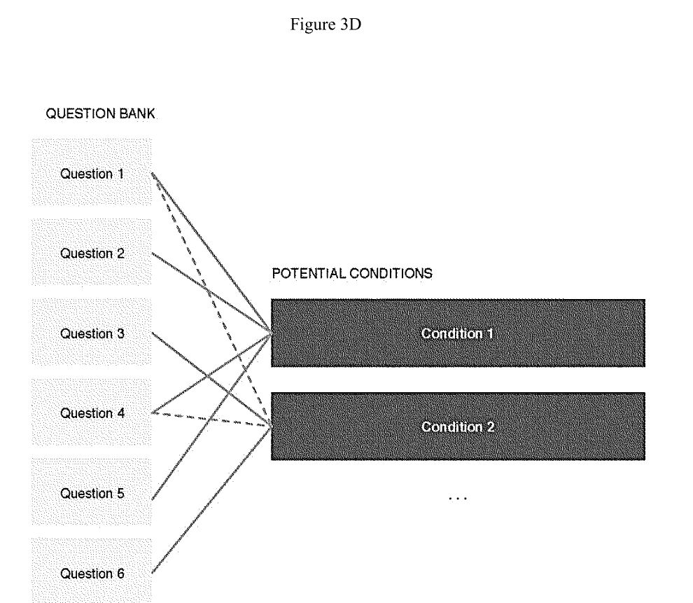

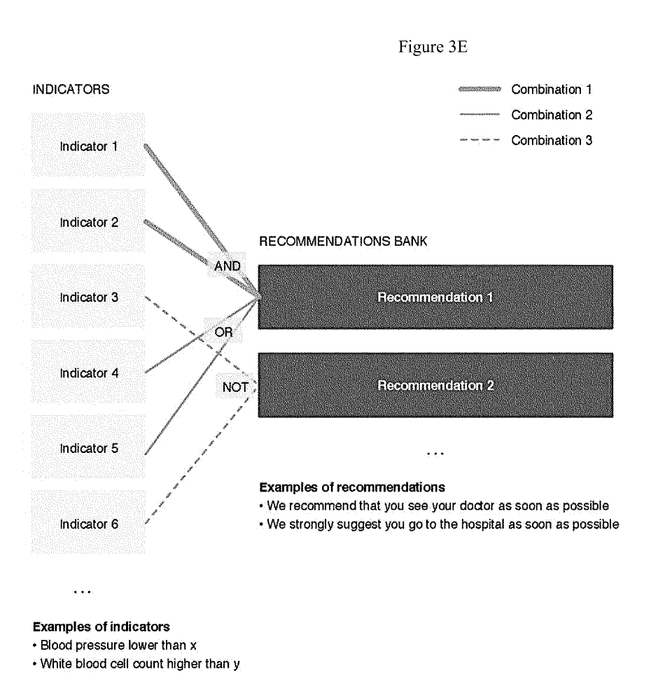

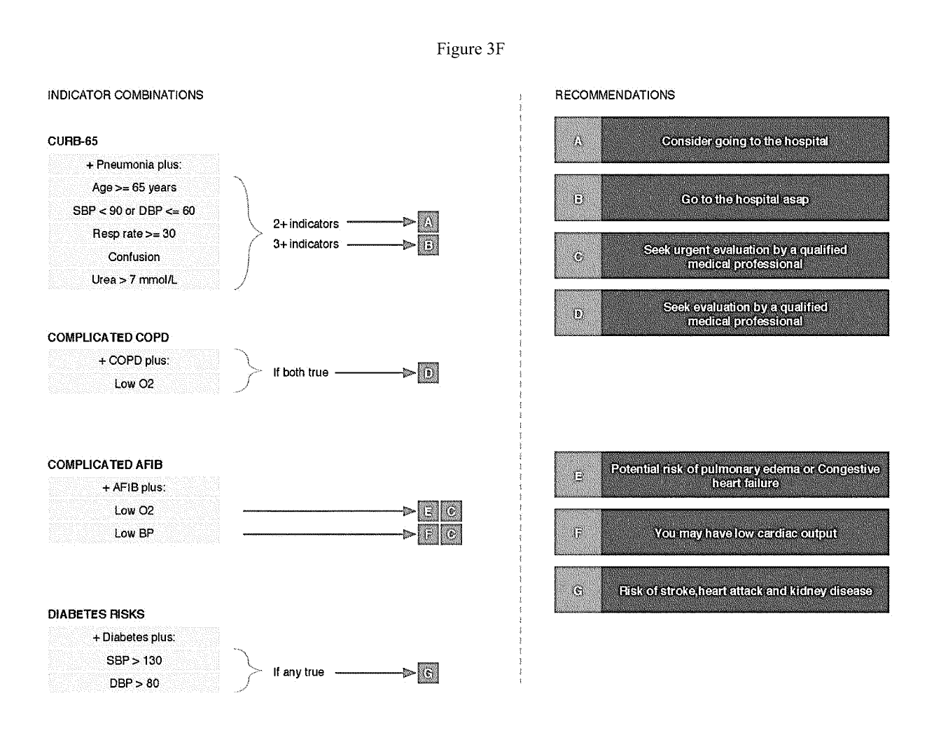

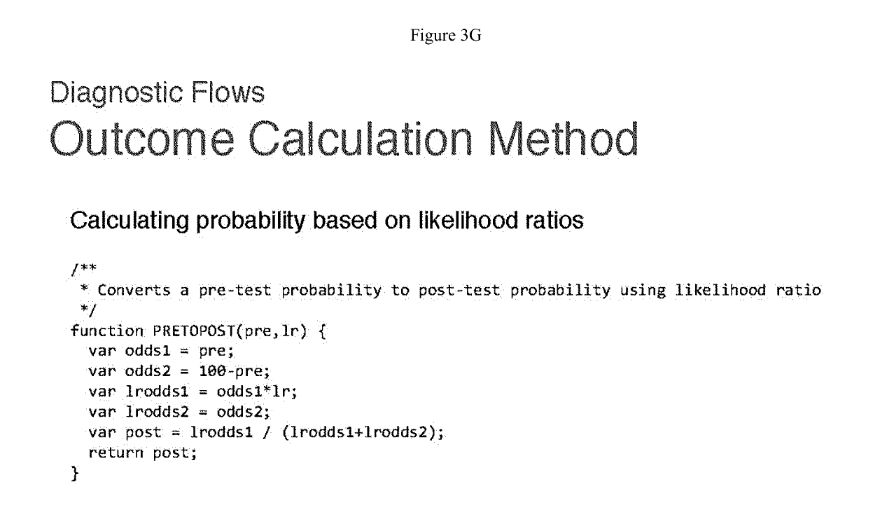

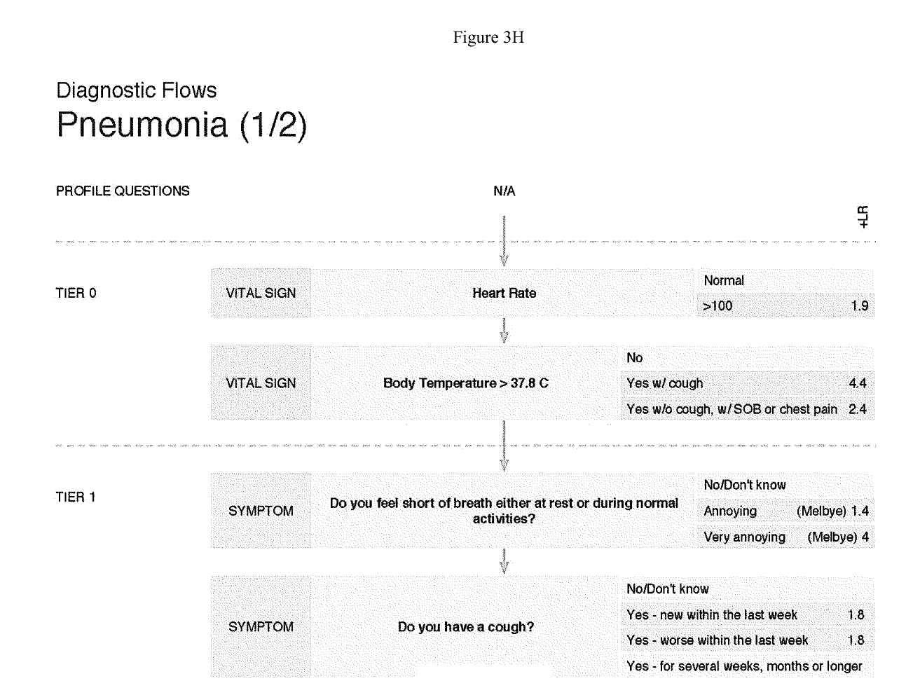

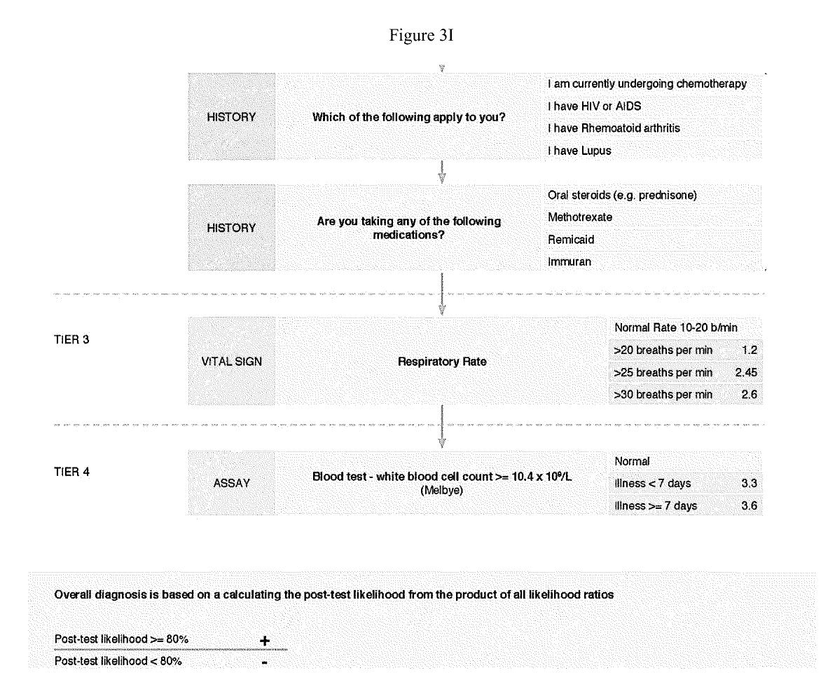

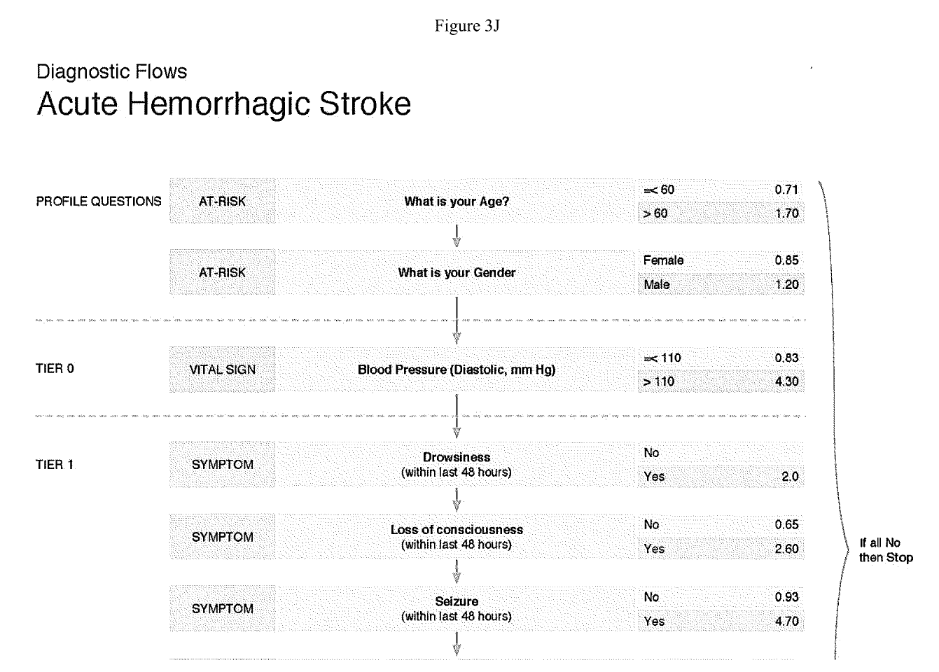

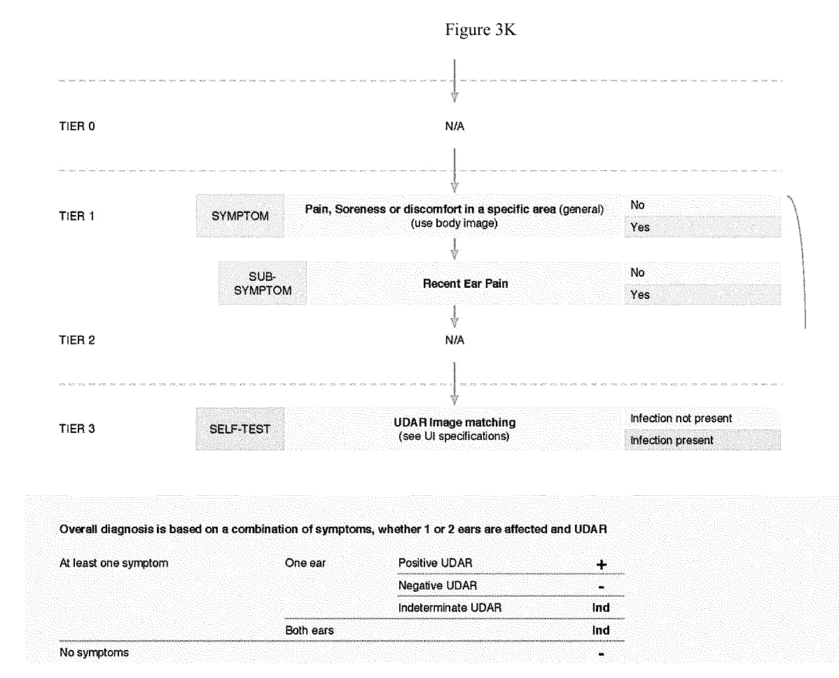

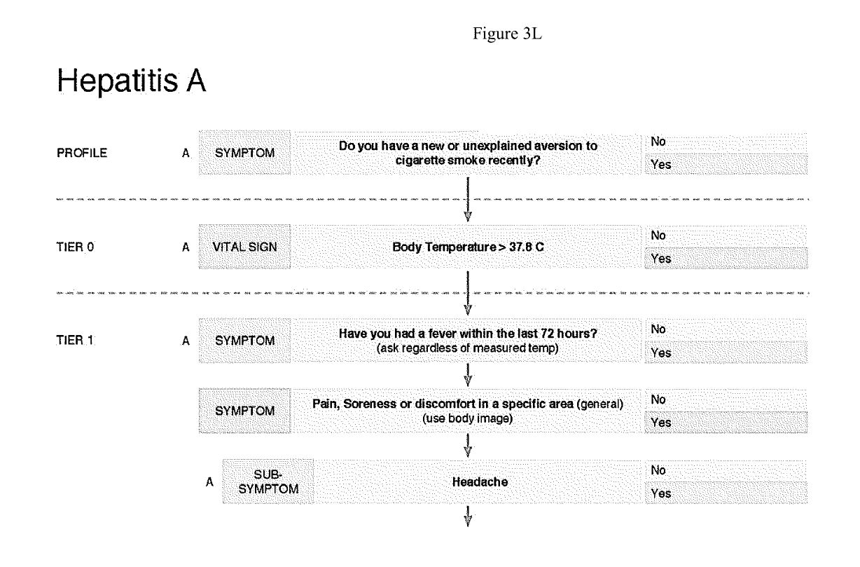

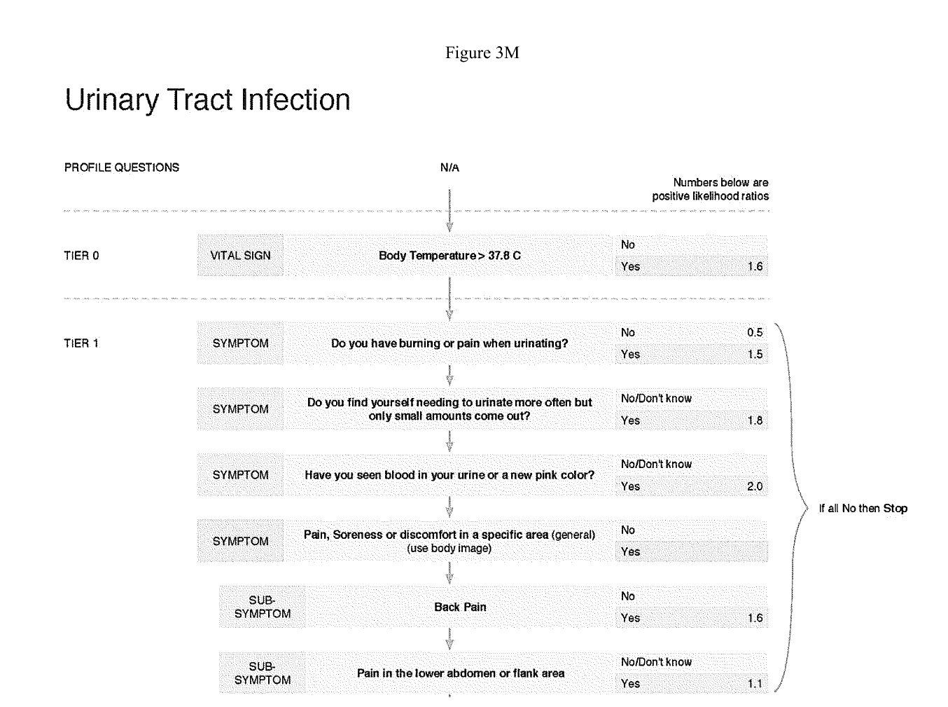

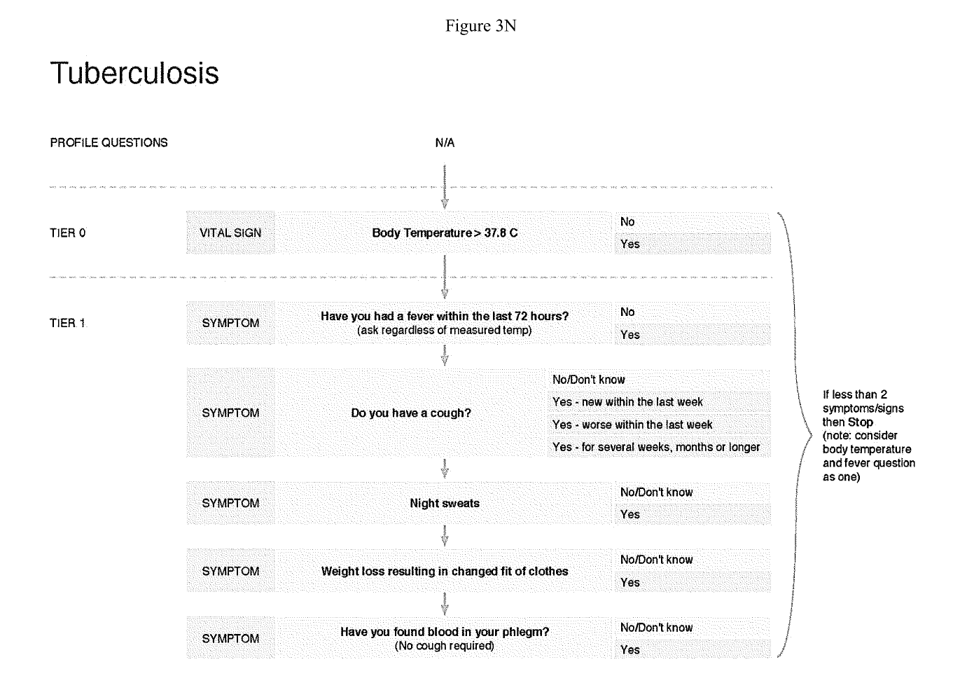

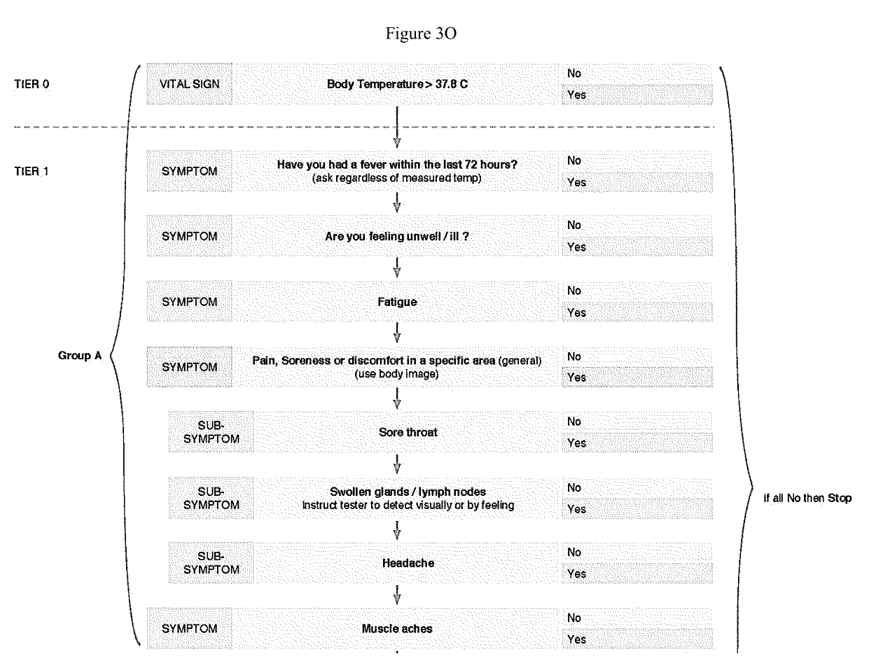

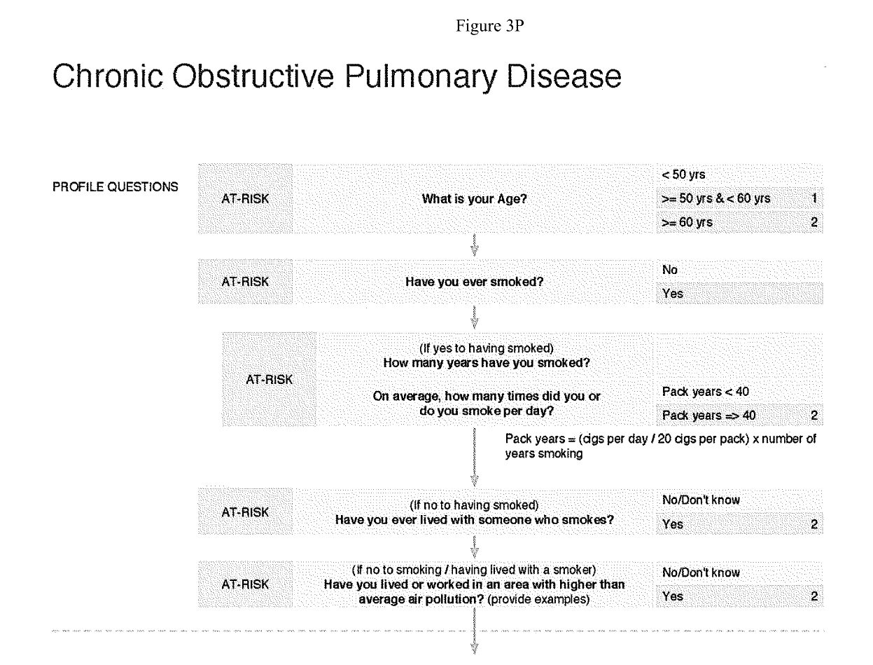

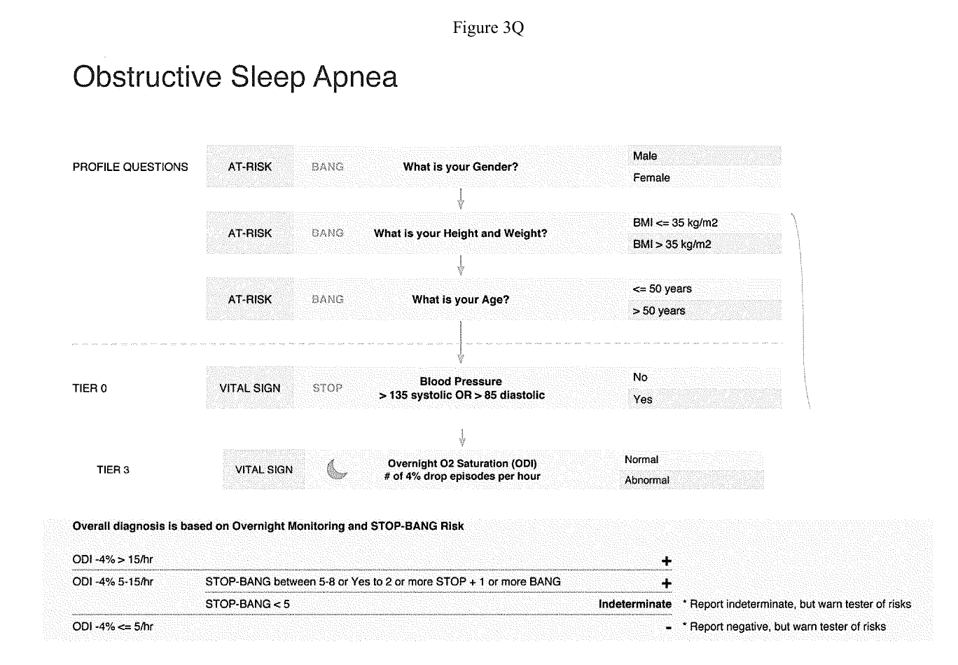

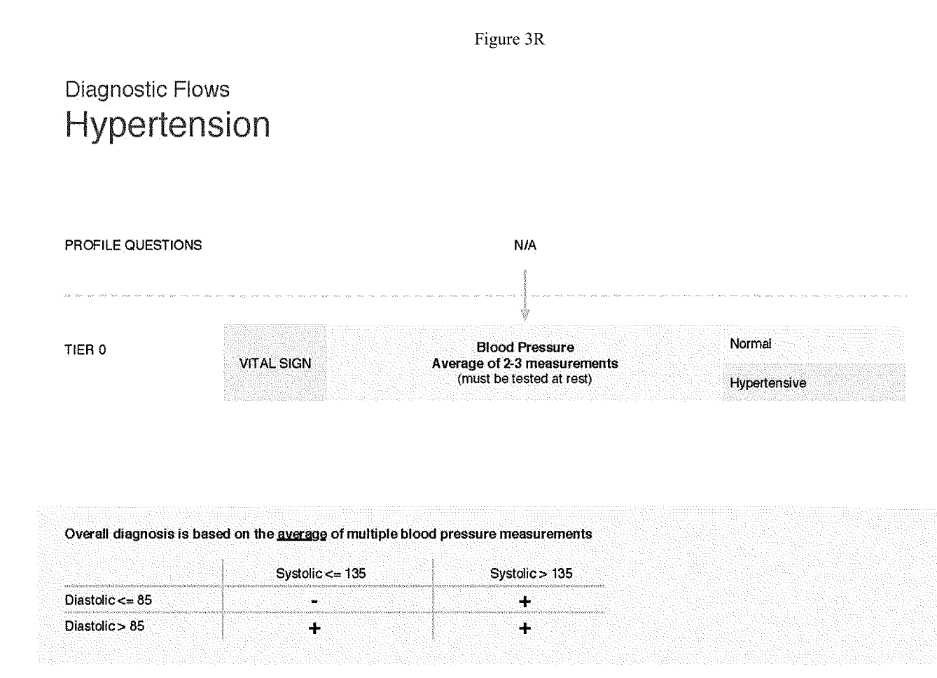

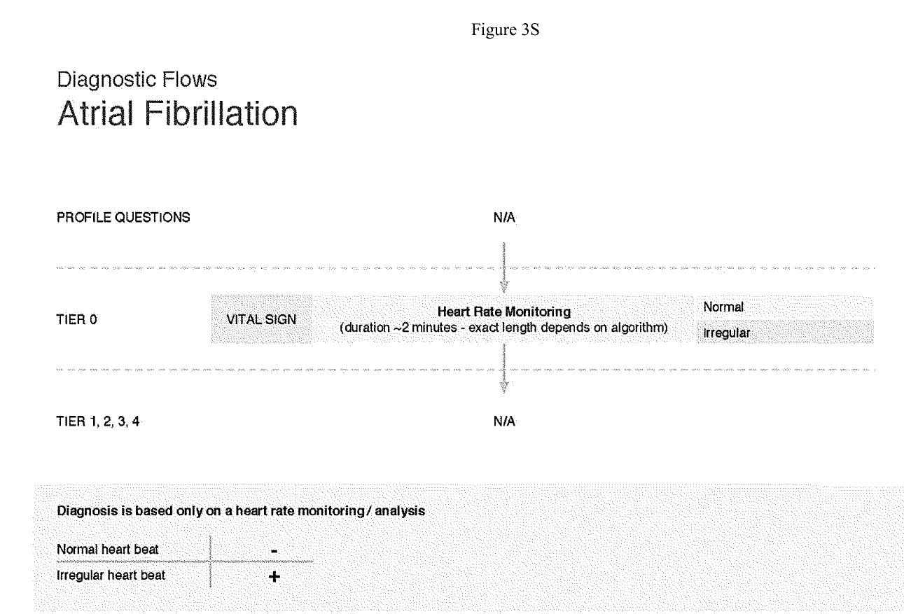

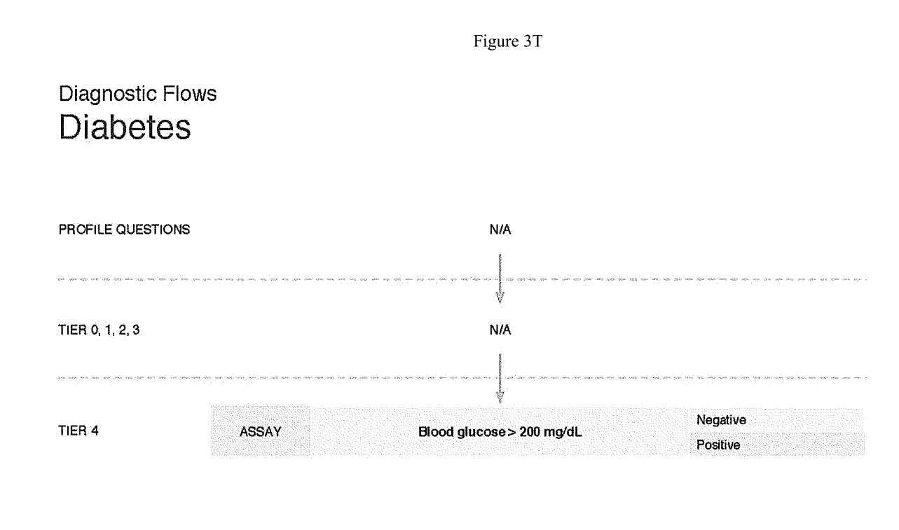

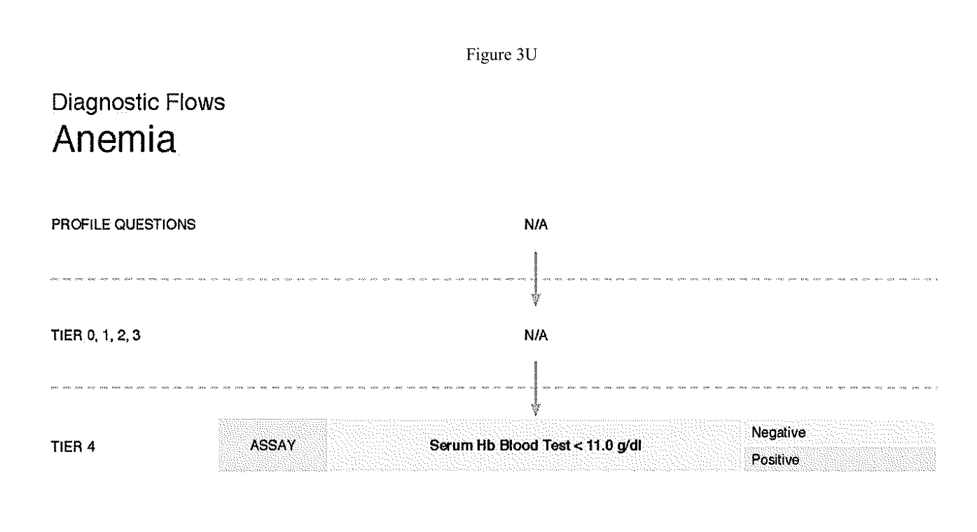

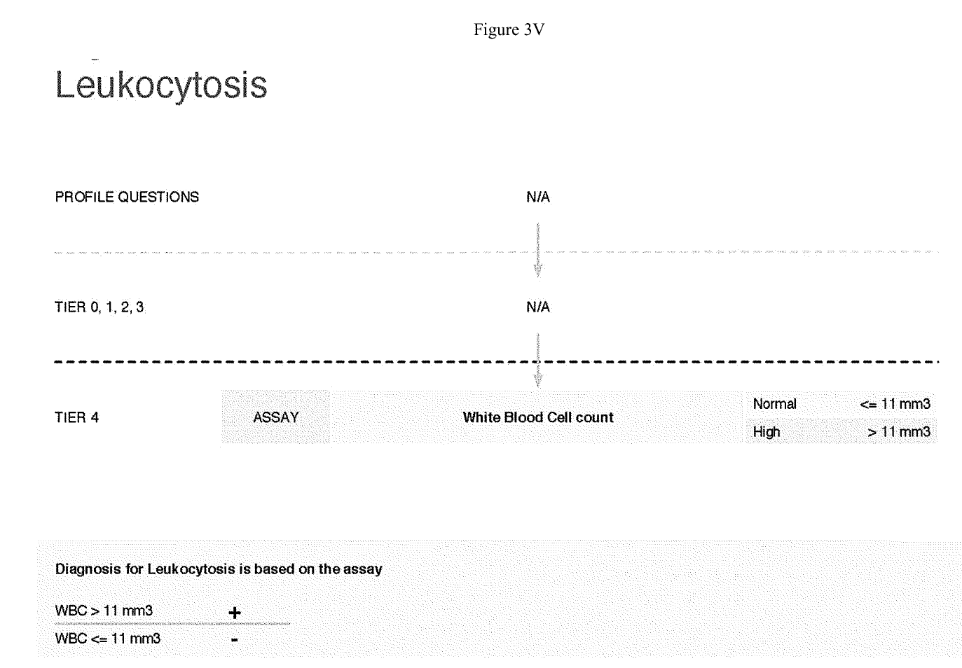

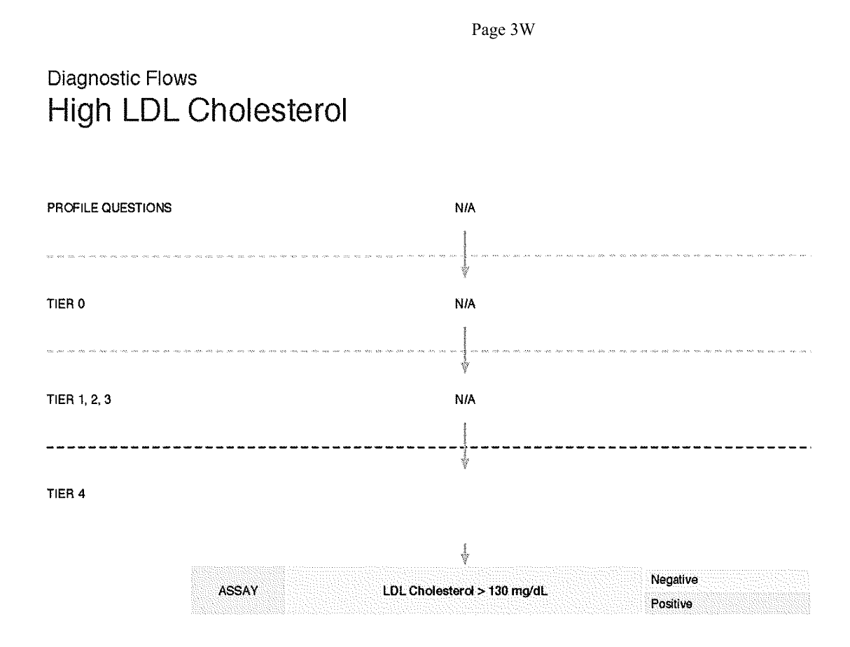

Examples of the software used to process and analyze ECG electrode signals is shown in FIG. 3S and elsewhere.

In other portions of the neck device, in order to help the device in a preferred yoke like shape, other portions of the neck device may contain rigid panels (404) to restrict bending in certain portions of the device, such as the arms, where it is desired that bending be restricted. These rigid covers may also be overmoulded with TPE, so that they are not repositionable.

Thus in some configurations, the neck device may allow for two areas of adjustment, such as: 1) a first major adjustment area near the rear of the device on both sides; and 2) a minor adjustment area on both sides near the front. This later adjustment area (area 2) can help ensure that latter area ensures accurate placement of the device's ECG electrode contacts in the standard medical V1 (4^{th} intercostal space, right of the user's sternum) and V2 (4^{th} intercostal space, left of the user's sternum) locations.

Thus this approach produces a wearable “yoke-like” electronic device formed from a posable armature encased on a soft material, while the addition of various rigid sections/covers acts to restrict or limit adjustment to only certain areas where such adjustment is desirable, while helping to prevent putting the neck device into undesired configurations.

### Use of Multiple, Hot-Swappable, Batteries to Allow Uninterrupted, Continuous Operation

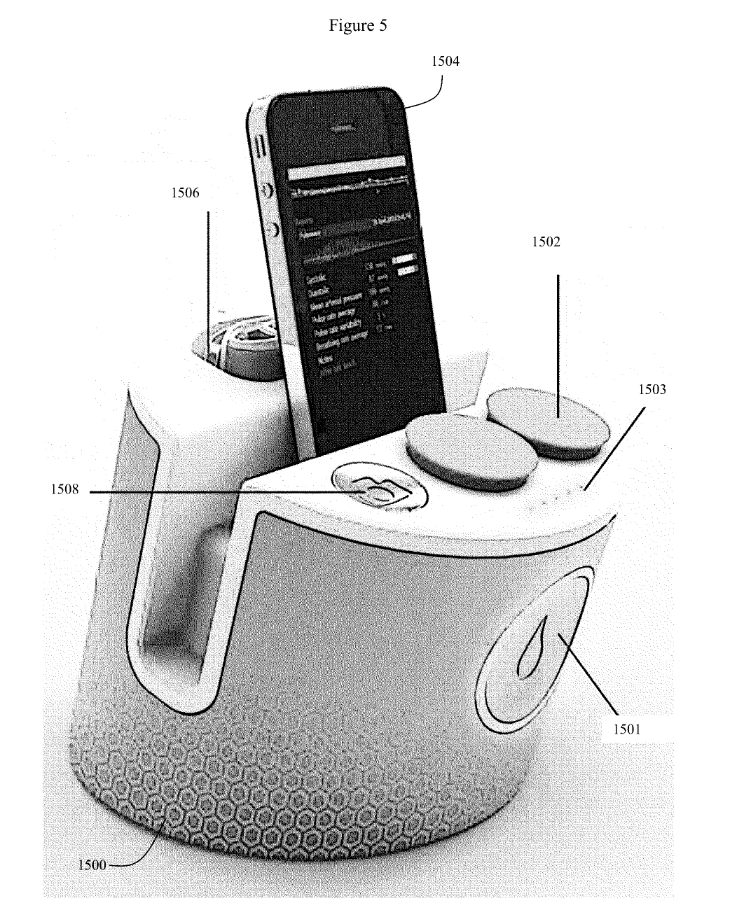

In some embodiments, the neck device may be formed using rechargeable batteries that can be either replaceable or non-replaceable, or charged in place. These rechargeable batteries (112, 114) may be charged by a temporary wired connection to an external power source (during recharging), induction recharging, and the like. However in a preferred embodiment, where tethering the user to a power source of any sort is not desired, the batteries can be removable and indeed may be hot-swappable (i.e. the device will continue to operate while batteries are exchanged). Here, a combination in-vitro diagnostic meter and battery recharger, such as shown in FIG. 5, or other type battery recharger may be used.

In this later embodiment, to ensure continuous operation of the neck device for extended periods of time, the neck device may be designed with multiple batteries configured to allow for quick removal (when the batteries are depleted), and easily replacement by fully charged batteries. The device can either maintain some non-removable batteries, super capacitors, or be designed so that it continues to operate using power from a first removable battery while a second removable battery is replaced, and vice versa. Any of these arrangements will allow the device to continue to collect and analyze captured vital signs during the battery exchange process.

In some embodiments, the neck device may comprise three batteries. Two batteries may be located on the chest area (112, 114) (such as above the ECG electrodes 200, 202) and may be configured to be easily removed and replaced by the user. The neck device may also have a third battery, which may be permanently enclosed in the neck device (and often located in the rear of the device such as 400) which can be used to keep the neck device operating in case the two removable batteries are both simultaneously removed or depleted. The neck device may optionally further comprise battery cavities with spring contacts to maintain electrical conductivity with the removable batteries.

In some embodiments, the neck device may also use a charging and power management circuit designed to allow for multiple charge and discharge modes. The charging and power management circuit can be configured to ensure that the various batteries are charging and discharging as desired, such as charging/discharge evenly, or charging/discharge one after another. This charging and power management circuit can allow for flexibility with how the user can replace the batteries. For example, when the system is configured to allow all batteries to charge and discharge evenly, the hot-swap batteries would last for the longest duration, but would also need to be replaced together. By contrast, when the system is configured to charge/discharge batteries in a one after another manner, then one hot-swap battery could be replaced when it is depleted, while the device begins to start discharging the remaining hot swap battery. In some embodiments, as previously discussed, it may be useful to include a wire connection to an external charger, such as a USB family type port, to allow the user to charge all wearable batteries at the same time while the neck device is not being used. Such a USB family port could also be used to download stored data from the device, as well as program the device, at least in cases where wireless data exchange is not desired.

In some embodiments, the neck device may be packaged as part of a kit with at least four hot swap batteries. Here the kit instructions for use may advice that two such batteries may be used for powering the neck device while the remaining batteries can be charging on a separate recharging station.

In the embodiments shown in the various figures, the design of the hot swap batteries was driven by ease-of use considerations. Round shapes were used for these examples because such round shapes are generally more battery orientation agnostic (e.g. not requiring the user to pay precise attention to battery orientation). Additionally, when the neck device is worn under a shirt (most frequent envisioned use), where it cannot be easily seen, the round shape of the battery and neck device battery chambers are convenient. Such shapes allow the user to locate the batteries on the neck device, even with no direct visual feedback.

In some embodiments, the neck device battery chamber (500) may be constructed with various magnetic positioning devices (e.g. magnets) oriented to provide feedback to the user that the battery has been installed properly. Such use of magnetic positioning devices can help keep the batteries in place even when the user is vigorously exercising.

In order to allow the user to monitor battery levels, the charging states of the neck device's various hot-swap batteries can be represented by various methods. This can be done via direct display on the device itself (e.g. using LEDs or an LCD display), by a vibration actuator that can make the neck device vibrate when battery levels are low, by wireless output to external computerized devices such as smartphones, and the like.

Thus to reiterate, the advantages of the hot swap battery embodiment is that the multiple hot swap batteries can allows for continuous, uninterrupted vital sign monitoring and capture over extended time periods. The neck device can be configured to manage these hot-swap batteries, and any internal non-swappable batteries, according to various different types of charge/discharge modes depending on the user's needs.

### Embodiments with a Wearable Earpiece Comprising One or More Vital Sign Sensors

In some embodiments, it may be desirable to configure the previously discussed neck device (100) with a wearable earpiece (110). This wearable earpiece may contain one or often a plurality of vital sign sensors. As will be discussed, in some embodiments, it will be useful to further provide wireless data transmission functionality to the neck device—wearable earpiece so that vital sign data from the earpiece (and optionally other vital sign data collected by the neck device as well) can be transmitted to local computerized devices (such as smartphones), and analyzed using appropriate software and vital sign user interfaces (UI).

The wearable earpiece device (110) may either use its own onboard rechargeable batteries, or alternatively derive power from the neck device's batteries (e.g. 112, 114) via a cable attachment (108) to the neck device (110). Similarly the wearable earpiece (110) can either have its own onboard processor and wireless transceiver, or alternatively it can share computational processor time and wireless transceiver data communications time with one or more processors and wireless transceivers located on the neck device (400). In this later case, a cable attachment (108) between the wearable earpiece (110) and the neck device (100) may also be used, and indeed the same cable attachment (108) may be used for to provide power, various control signals, and various data channels between the wearable earpiece (110) and the neck device (100).

Although the wearable earpiece may be used to implement multiple vital sign sensors without the neck device (here the wearable earpiece will generally provide its own power source, processor, and wireless transceiver) one of the advantages of combining the neck device with the wearable earpiece to produce a unitized combined embodiment is greater efficiency. That is, a combined neck device and wearable earpiece system can combine vital sign sensors from the neck device and the wearable earpiece into a unitized system. Thus system costs can be saved due to the containing multiple shared electronic components. Here common components such as the combined system's batteries, wireless modules, and processors can all be shared, and resources allocated as needed by the various sensors at various times. In a preferred embodiment, this combined neck device and wearable earpiece can also take advantage of commonly available personal computerized devices, such as smartphones (e.g. iOS, Android, Windows type smartphones), to implement additional processing and to provide various user interfaces (e.g. app based user interfaces) as desired.

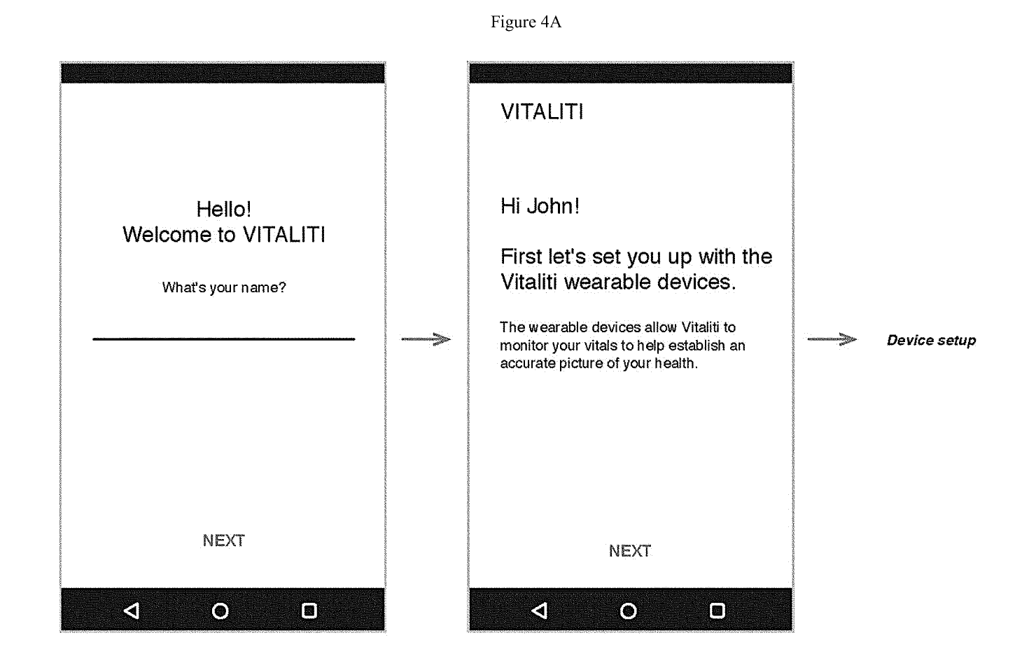

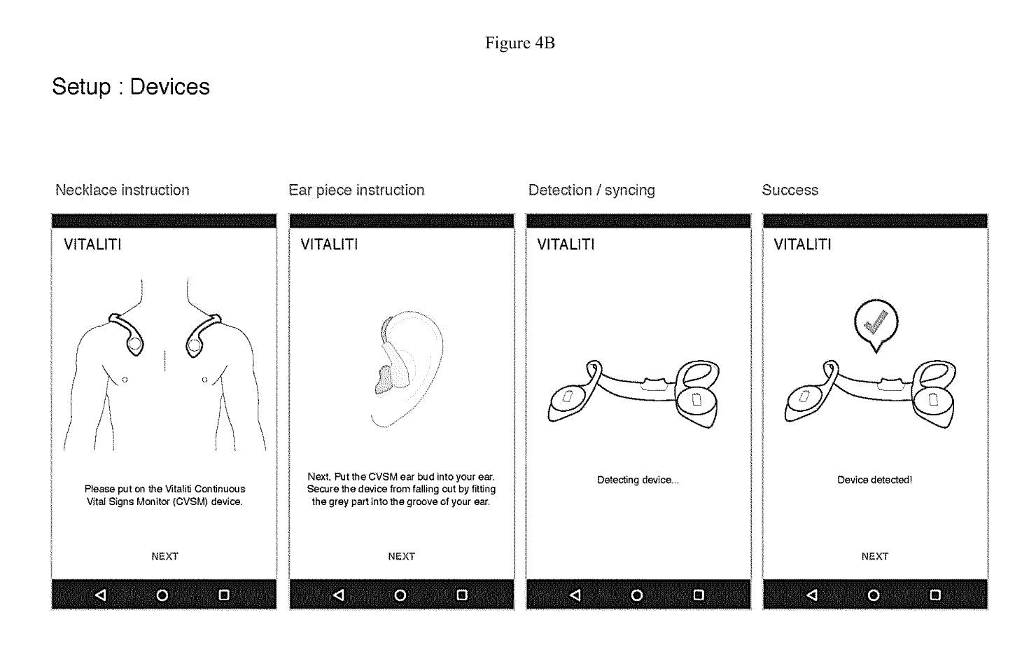

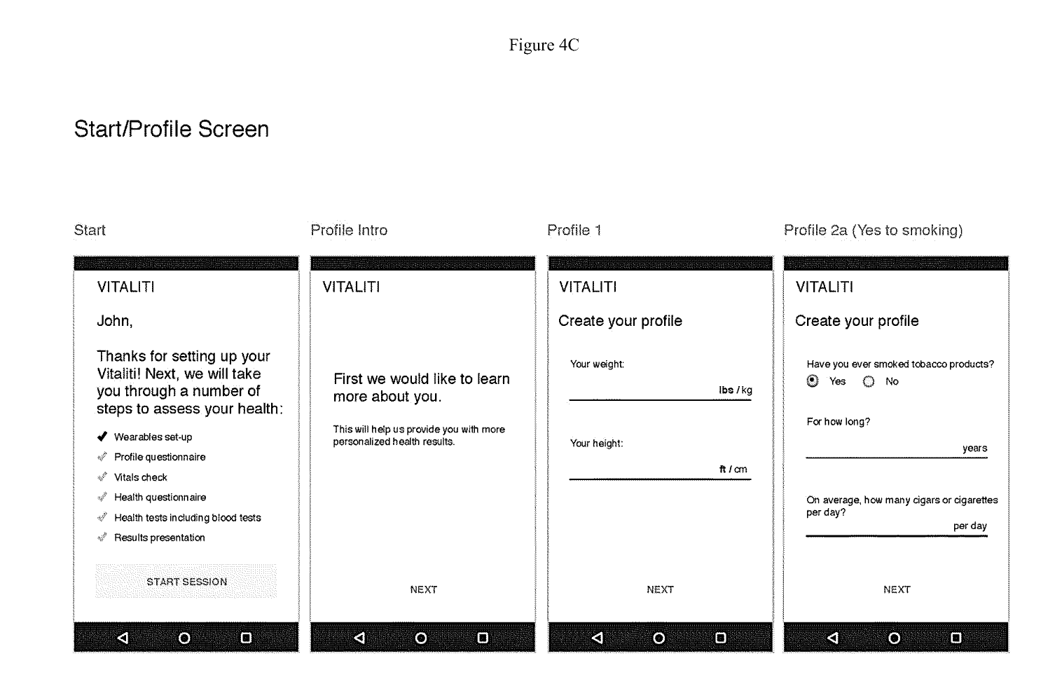

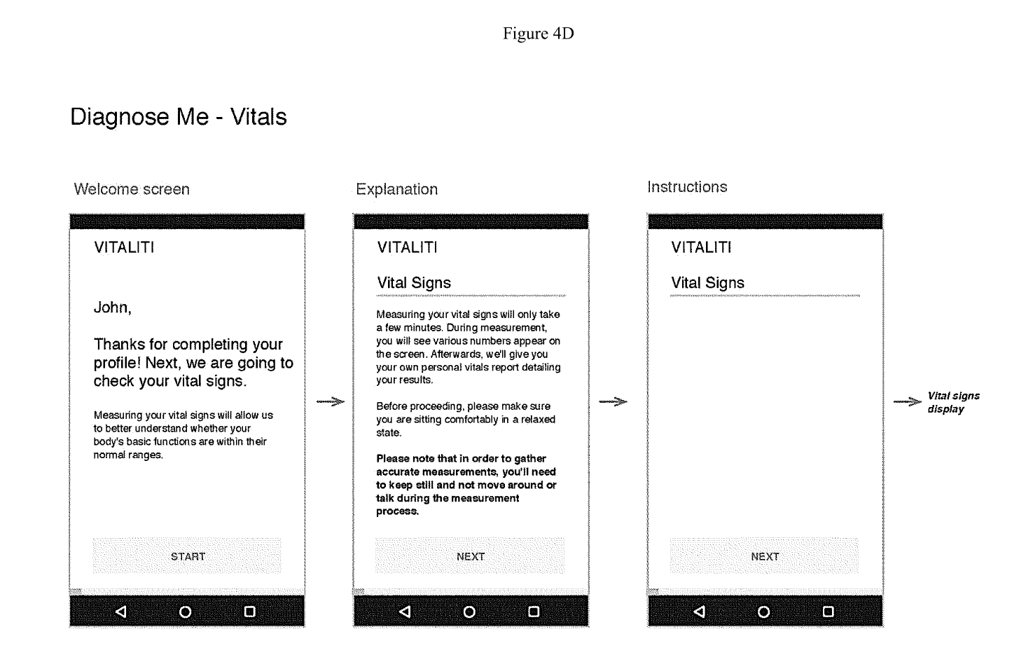

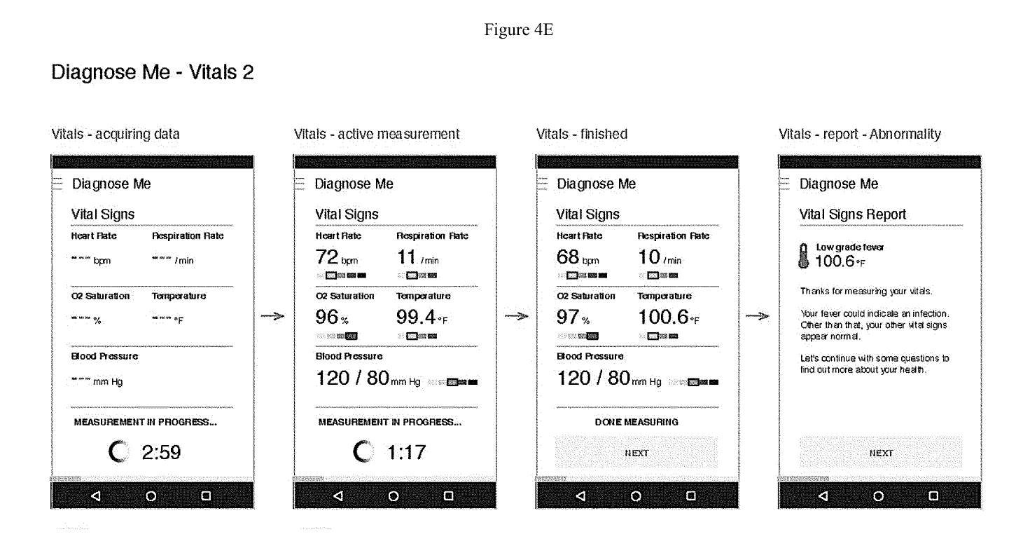

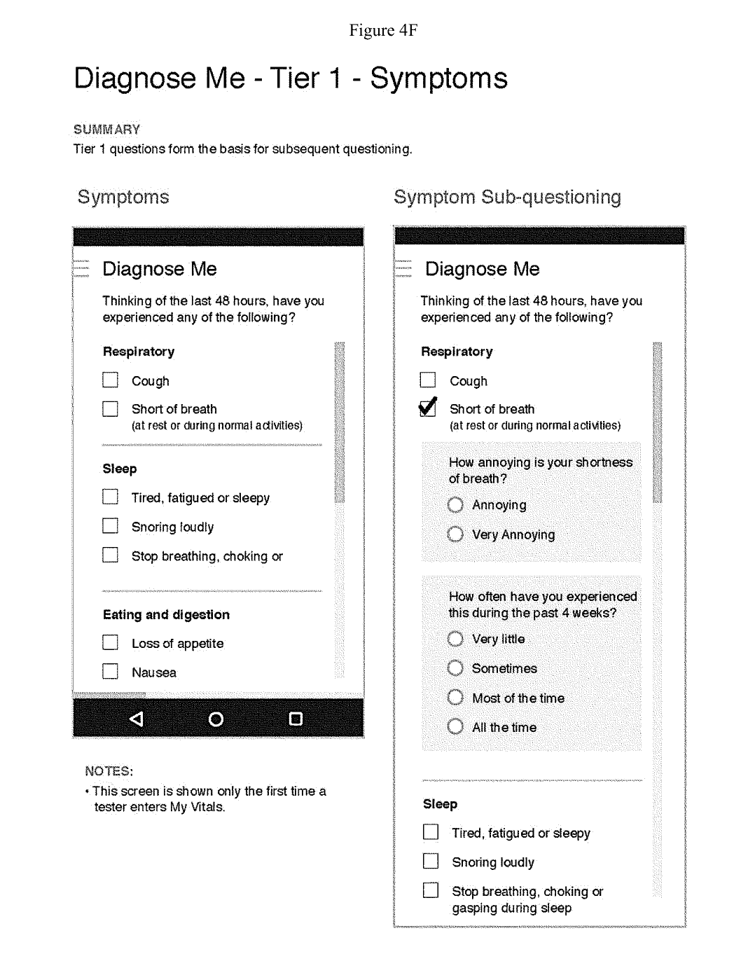

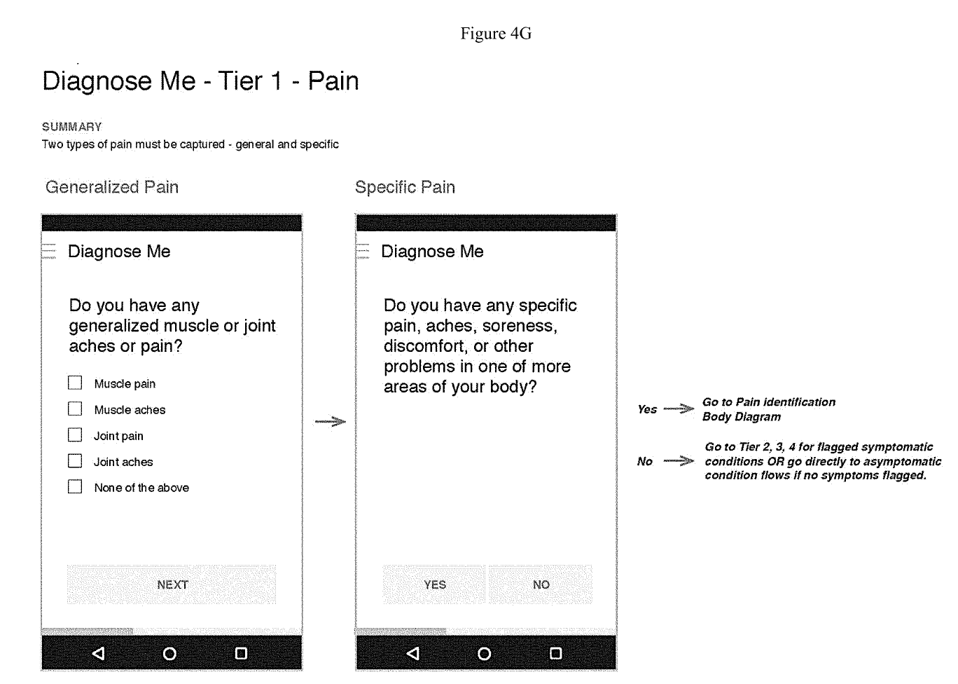

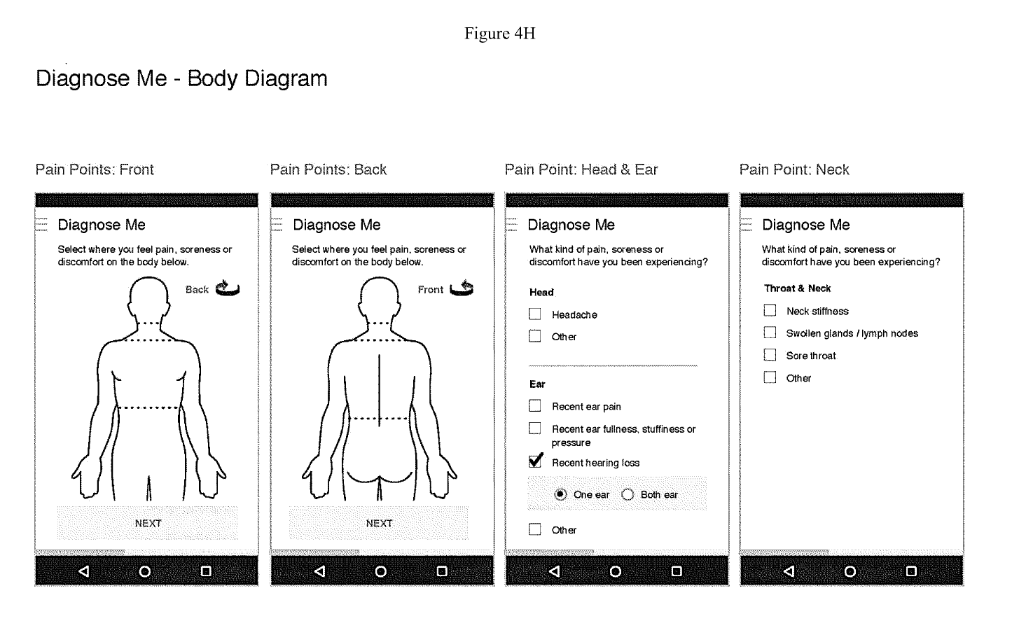

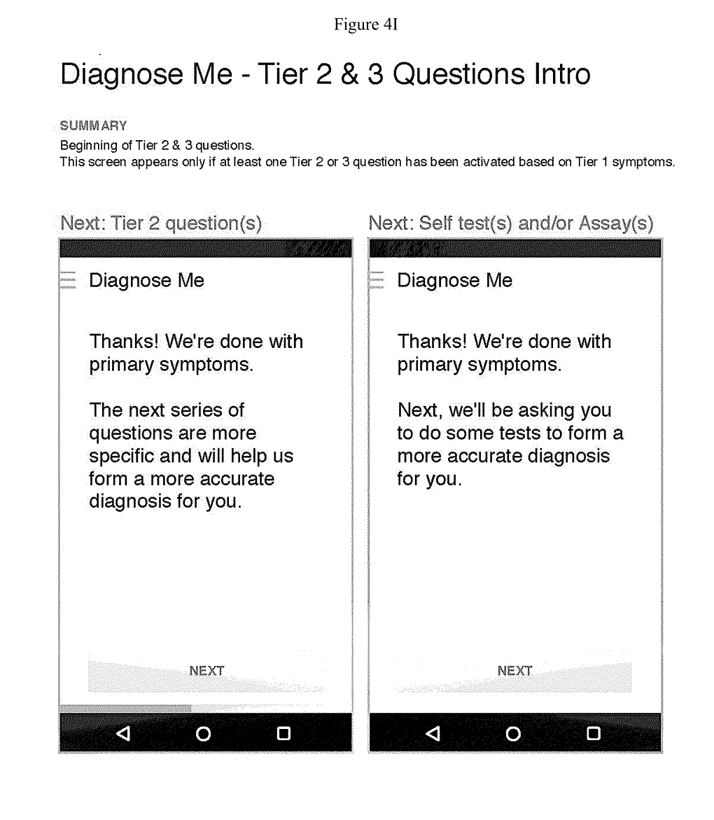

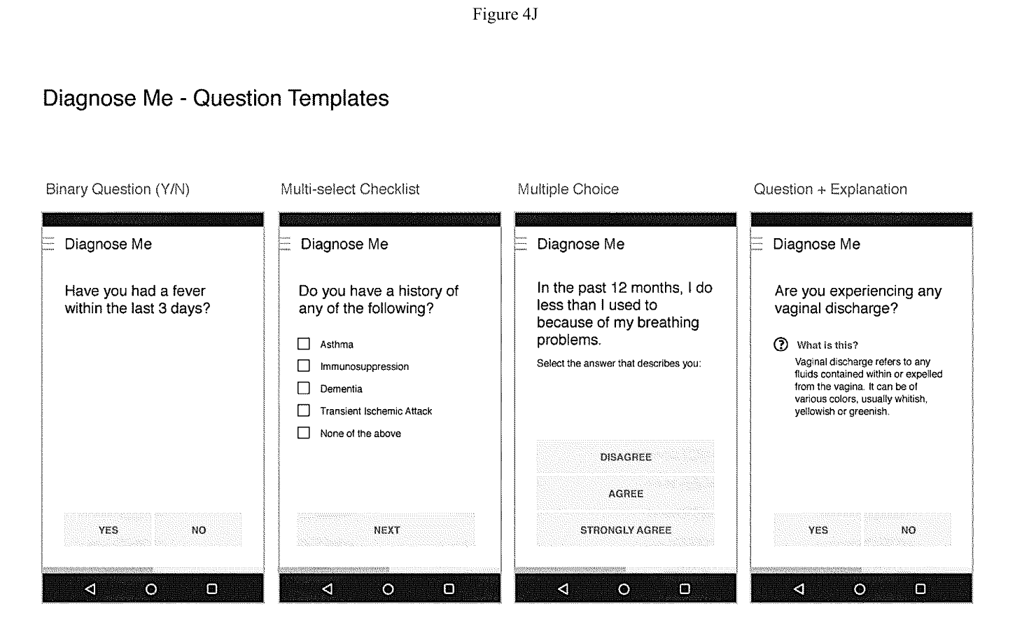

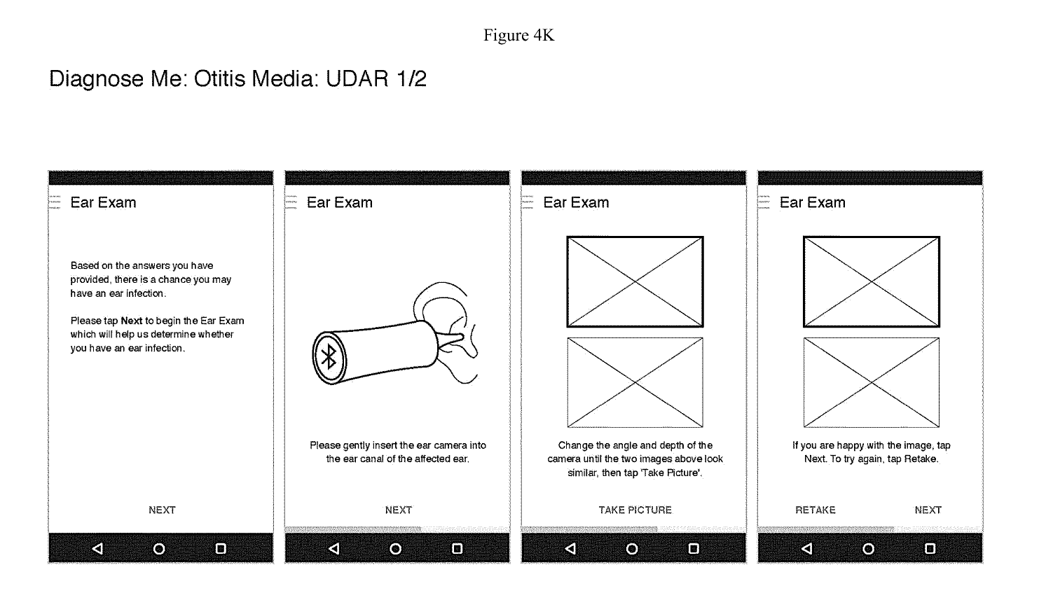

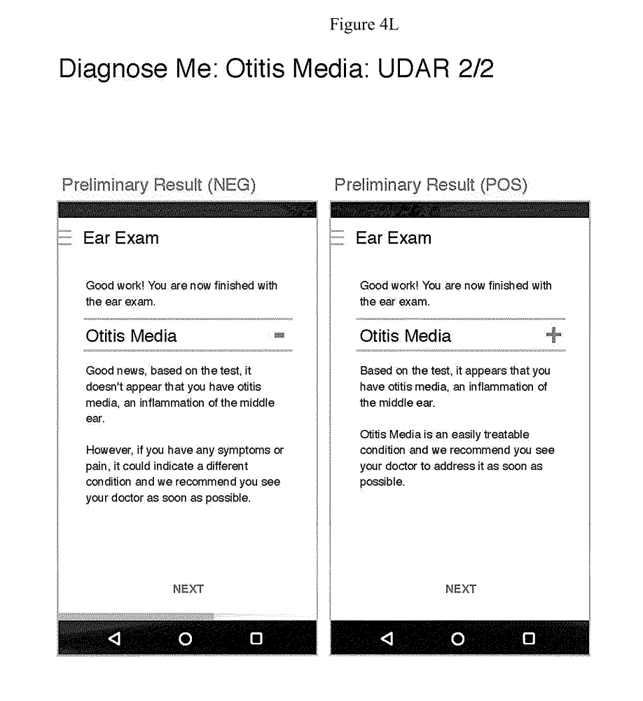

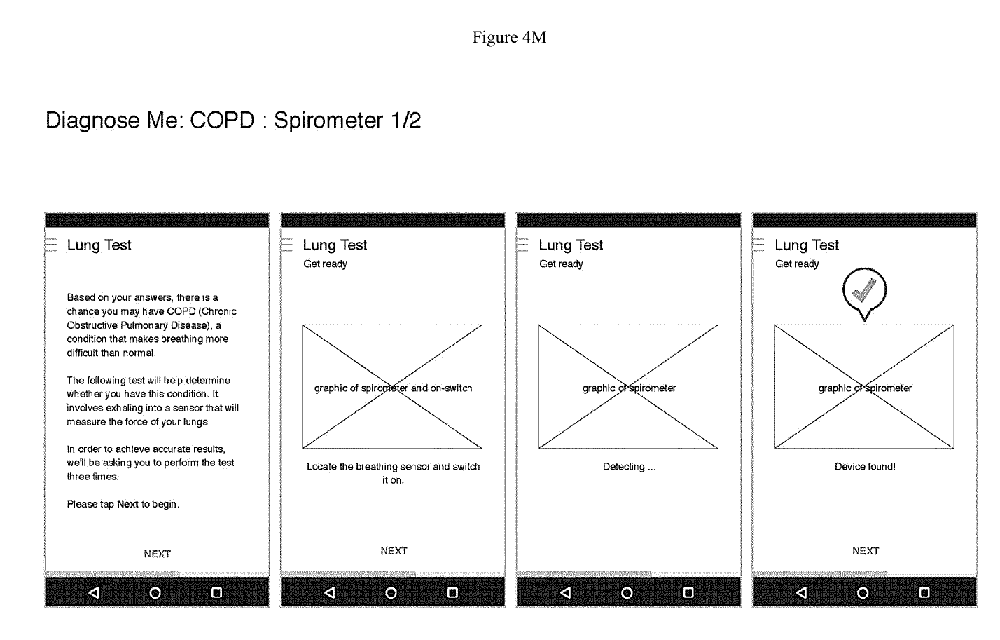

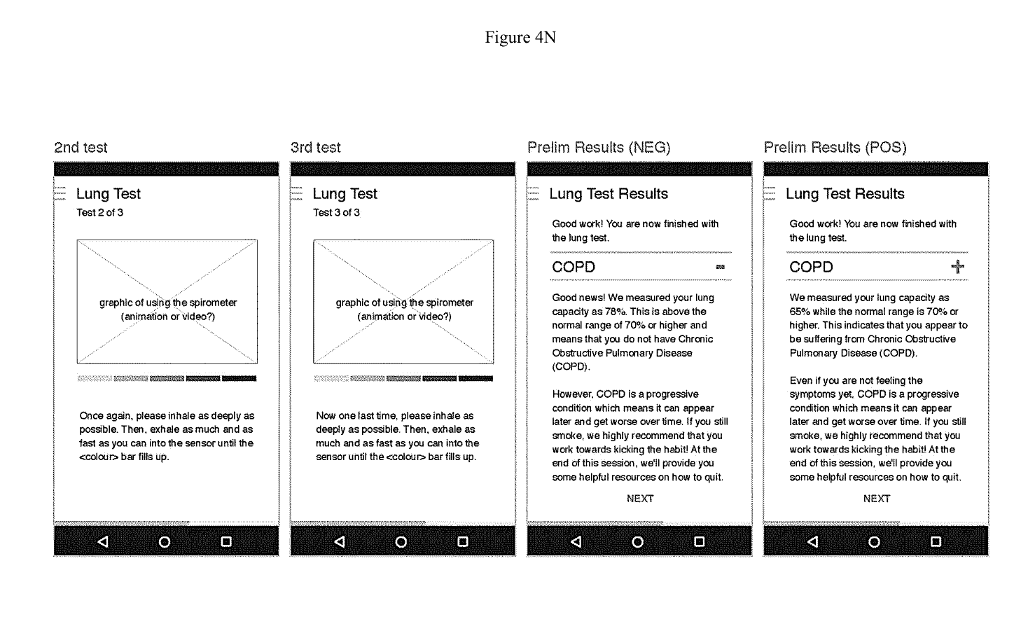

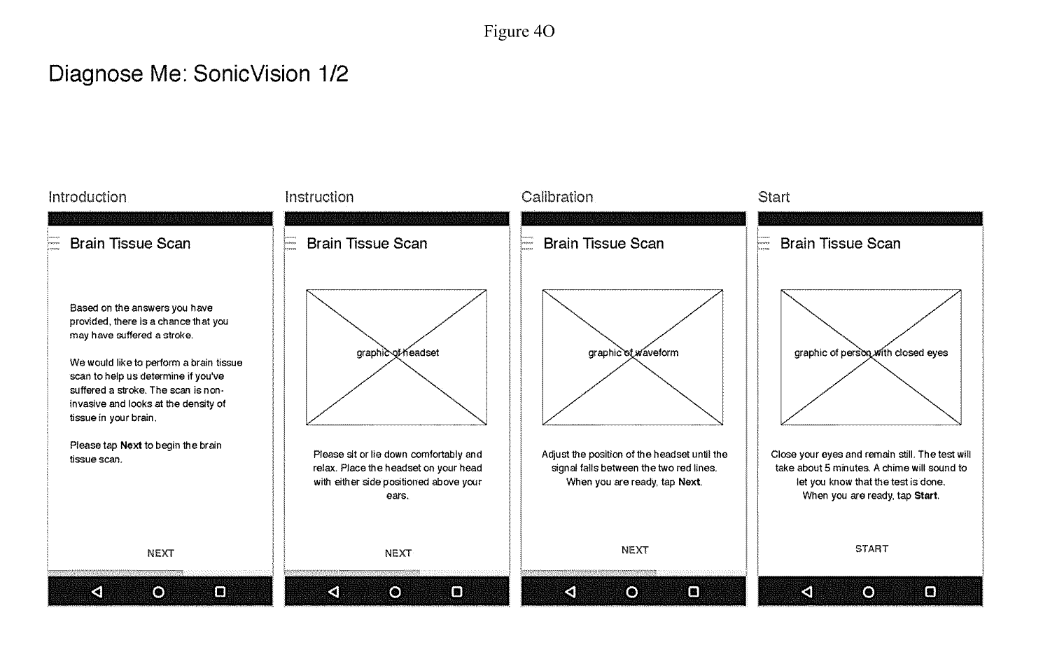

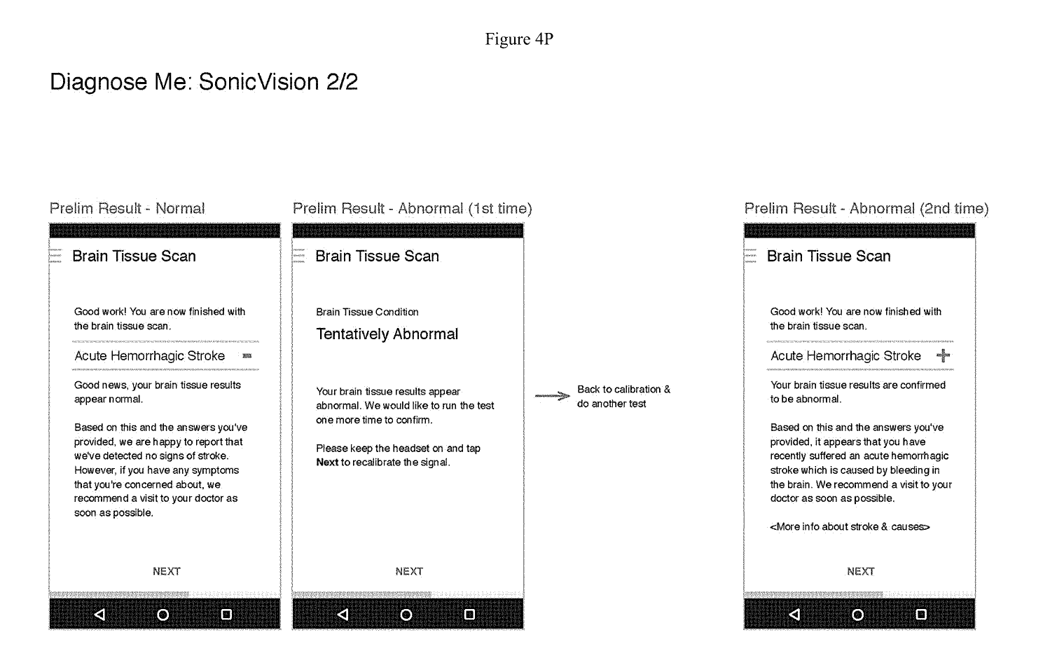

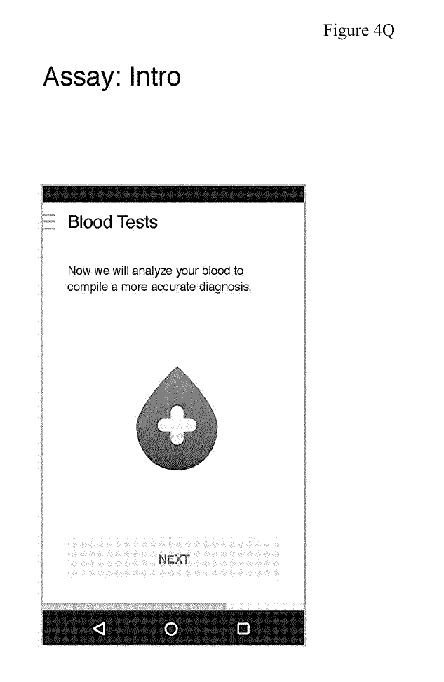

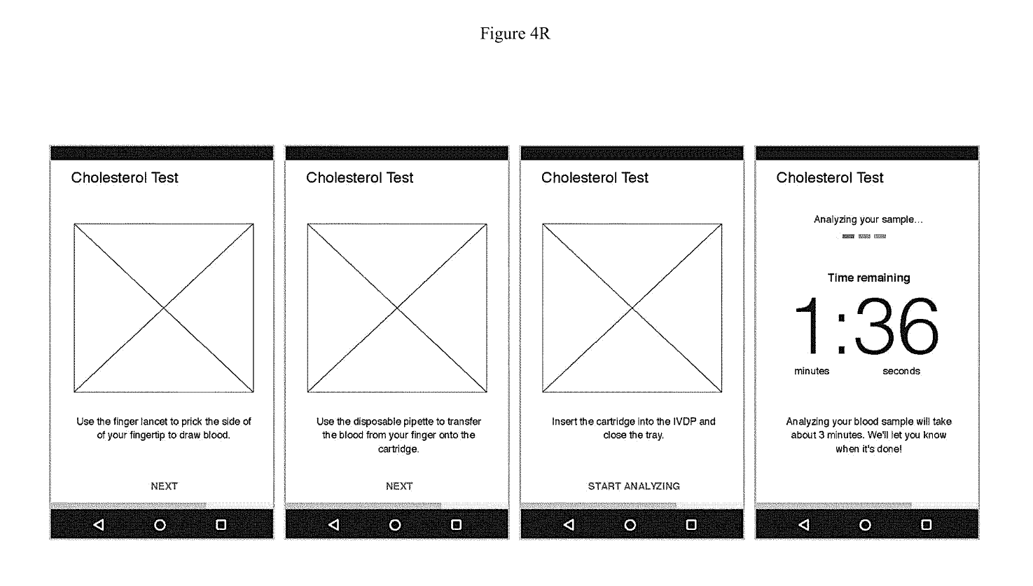

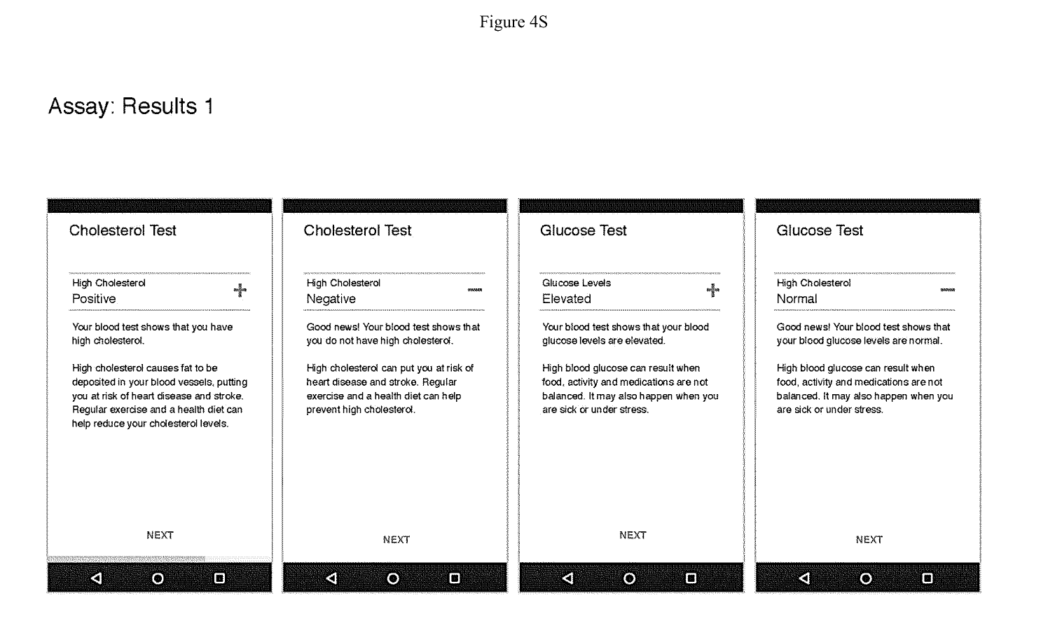

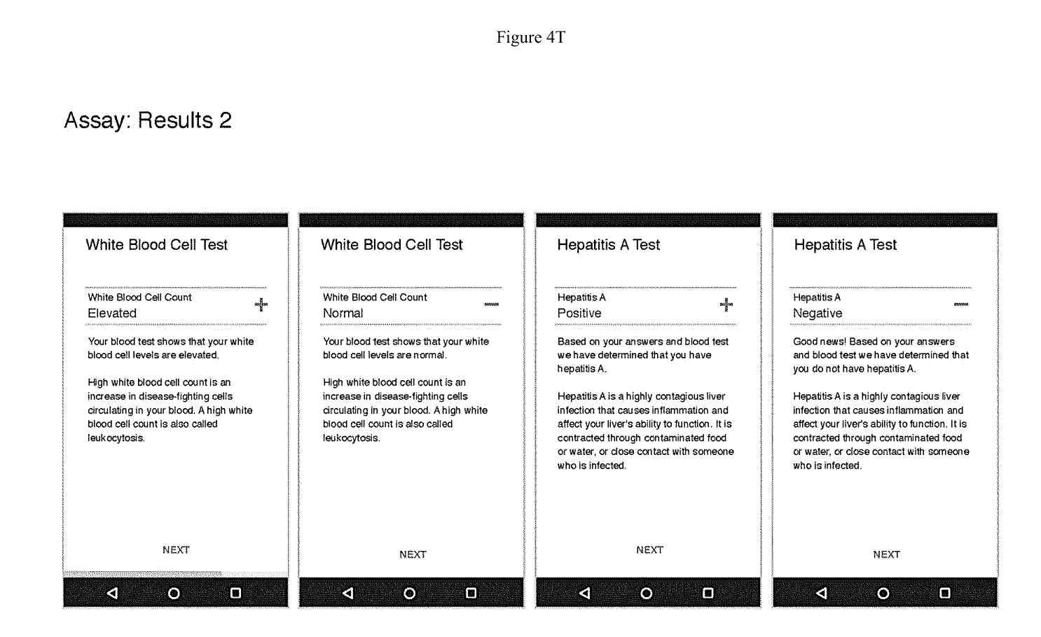

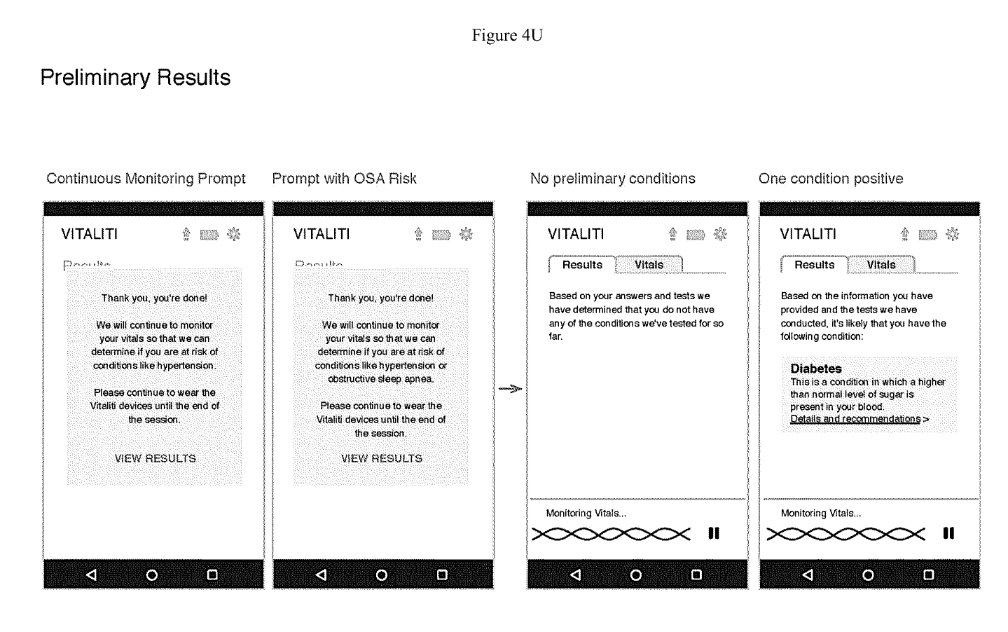

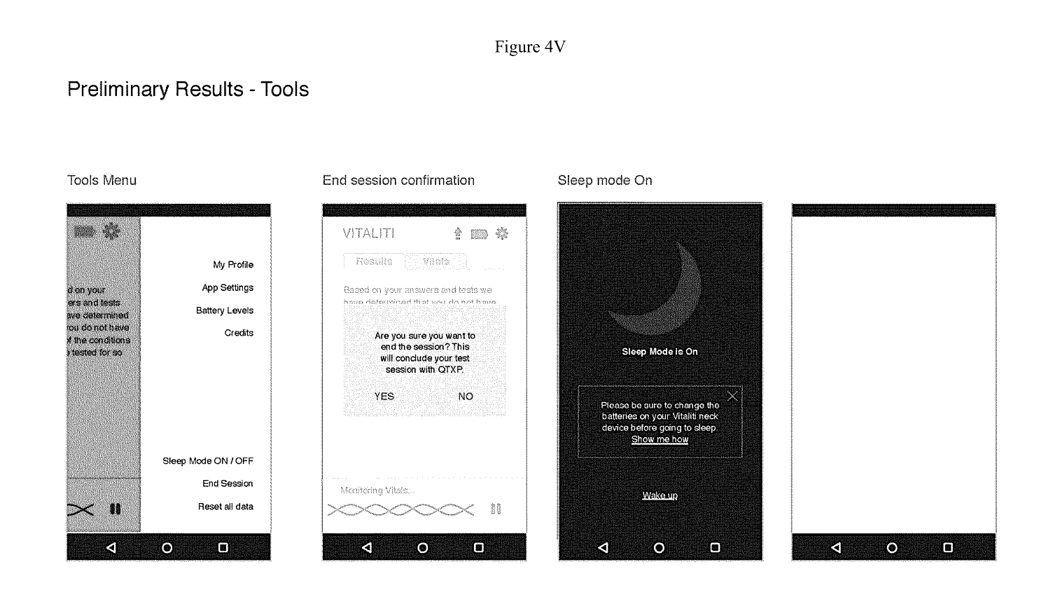

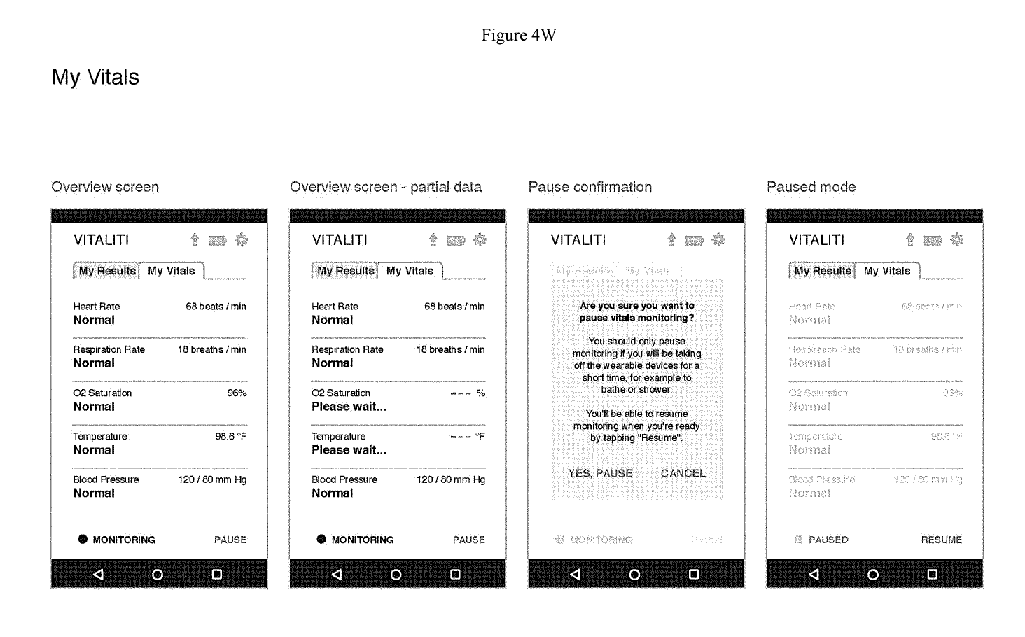

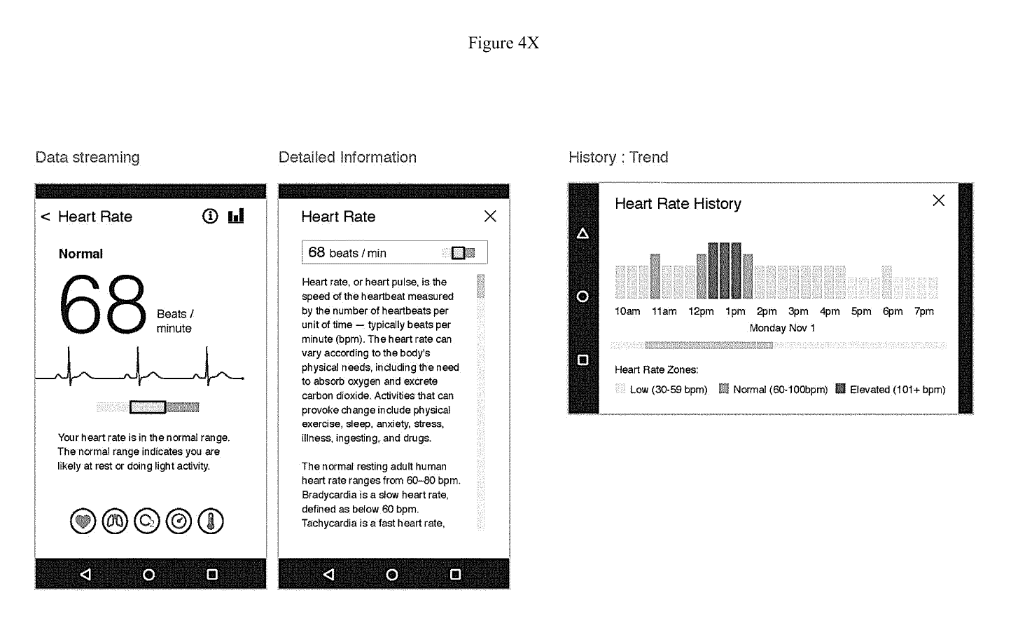

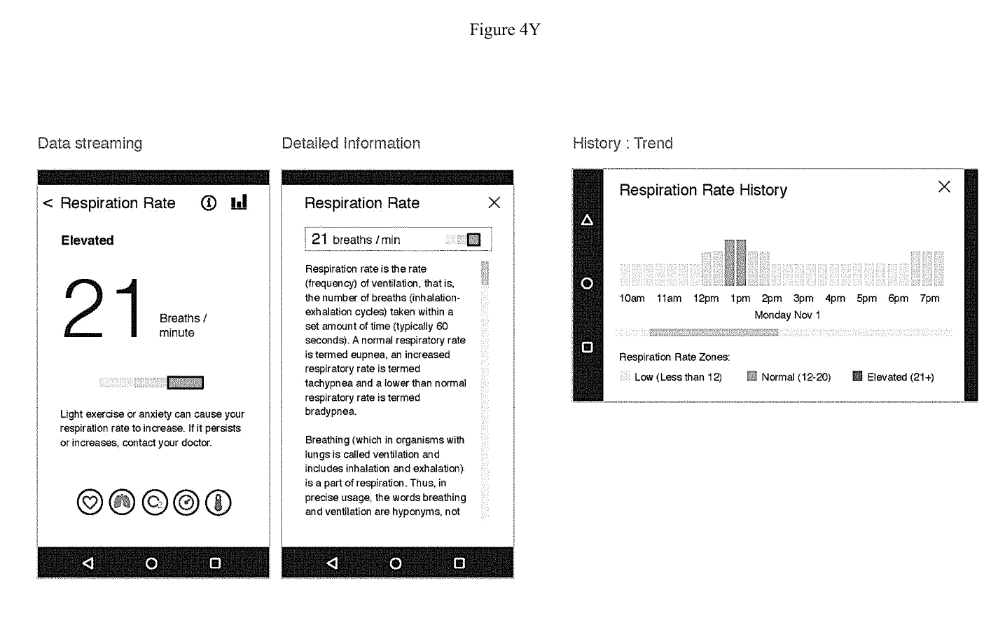

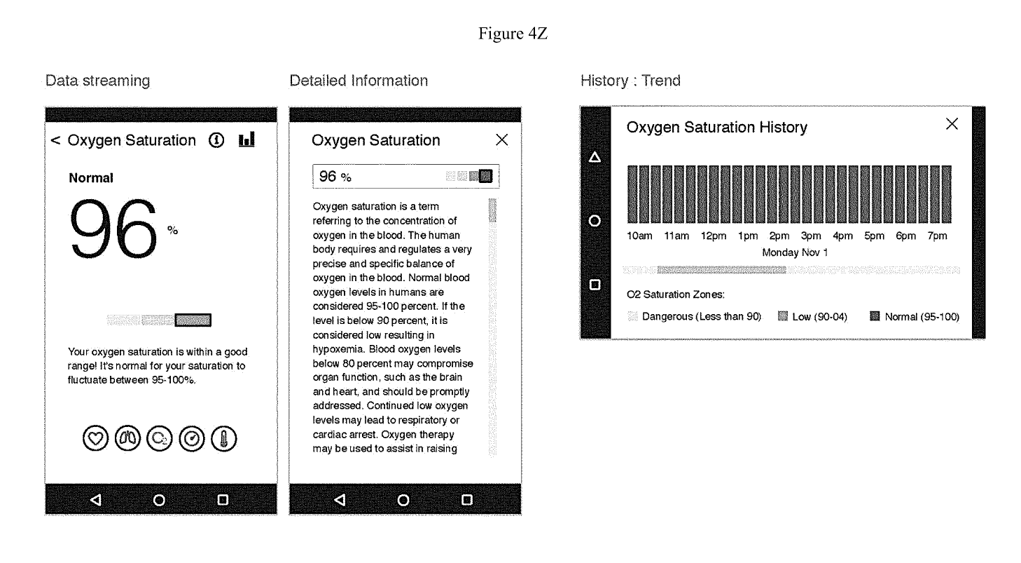

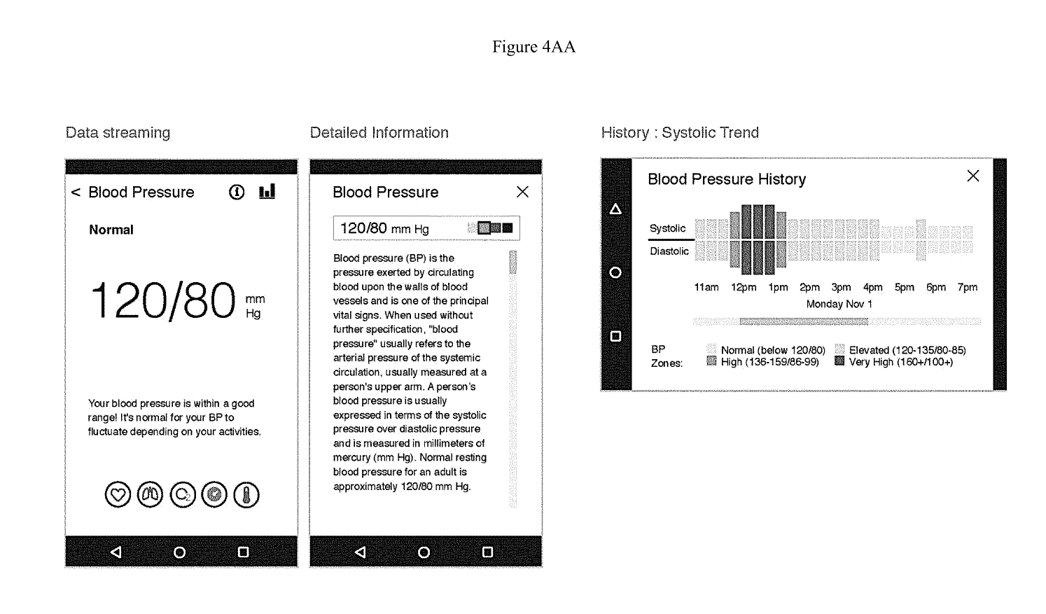

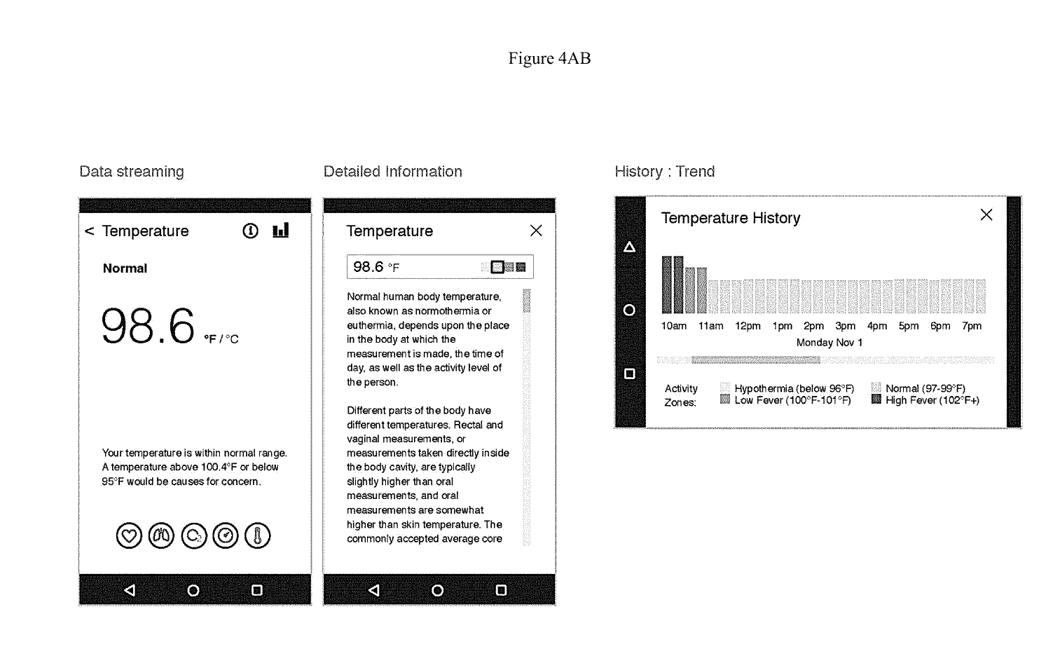

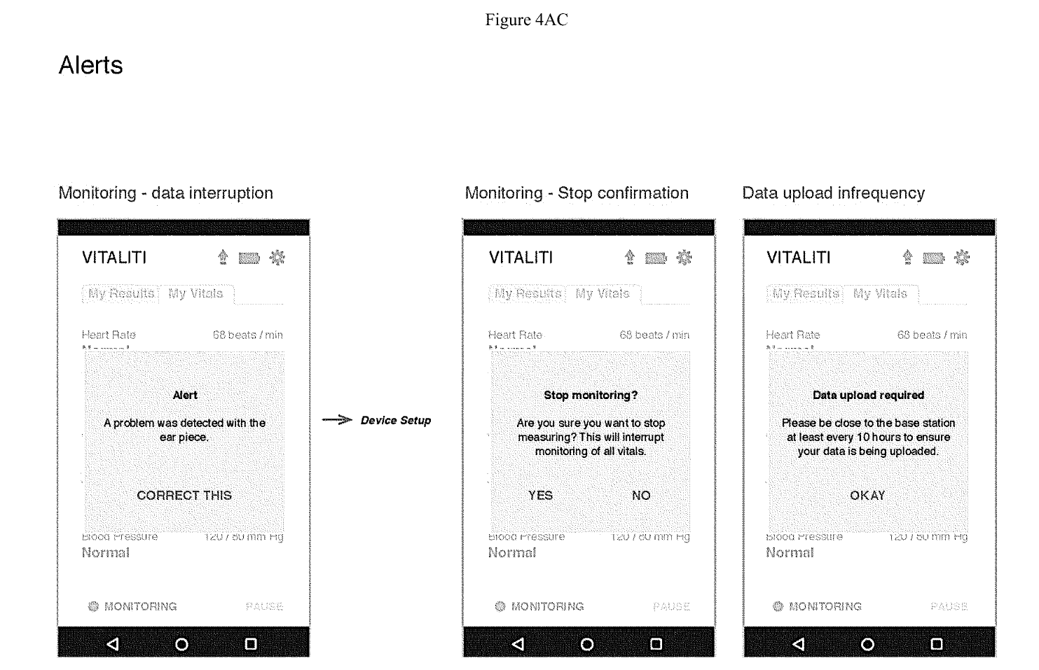

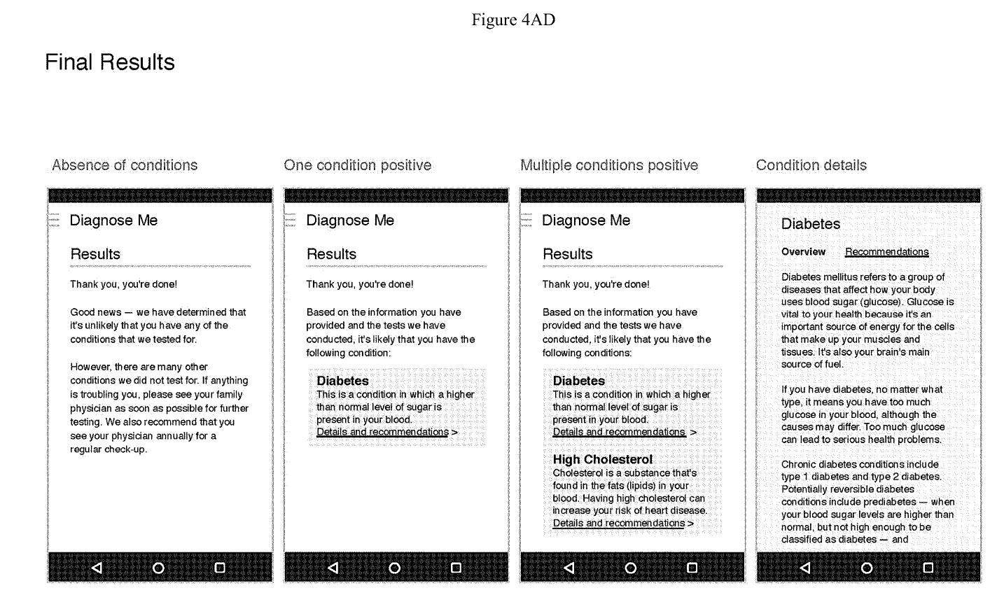

Use of external computerized devices such as smartphones to provide additional diagnostic processing capability, and also to provide a user interface for the user, is shown in more detail in FIGS. 4A to 4AD, and FIG. 5.

Thus in some embodiments, the ambulatory patient wearable computerized device may further comprise, or at least be configured to communicate with, a smartphone or tablet device. In these embodiments, the user interface may comprise a touch sensitive display screen, such as a smartphone or tablet touch sensitive display screen.

As will be discussed shortly, running the system's diagnostic algorithms (see FIGS. 3A to 3W) and user interface (see FIGS. 4A to 4D) on a local computerized device such as a smartphone has many advantages. In addition to reducing development complexity, the high capability (large touch sensitive, high resolution display screens, good processing power, high memory, and high connectivity) of modern smartphones allow for sophisticated management and processing of data from the various sensors. This approach also lends the device to larger markets in developing and 3rd world countries where cost-prohibitive medical equipment is unlikely to be purchased and deployed widely.

Wearable Earpieces Employing Photoplethysmography (PPG) and/or Body Core Sensors

Blood oxygen saturation, (SPO₂), is a vital sign that is critical in determining a patient's overall health. Prior art devices, such as blood oximeters, tended to monitor blood oxygen levels using traditional parts of the body such as the fingertips or earlobe. However the drawback of such prior art approaches is that wearing a fingertip or earlobe attached device for prolonged periods of time, for an ambulatory patient, tends to be both conspicuous and often uncomfortable. Thus less conspicuous and more comfortable approaches are desirable.

Thus in a preferred embodiment, the wearable earpiece device (110) may be configured to resemble less conspicuous and commonly worn earpiece devices, such as ear bud style earphones, hearing aids, ear plugs, and the like, and will usually not camp sensors onto the user's earlobe as is customary for blood oximeters.

Instead, the invention's wearable earpiece device may be configured with various types of sensors, such as PPG sensors and infrared temperature monitoring sensors.

For example, the earpiece (110) may position the optical and light sources (usually light emitting diodes) for the PPG/blood oximetry sensors so that they illuminate areas of the ear other than the earlobe, such as the concha and antitragus region of the external ear. This alternative portion of the ear has good inherent vascularity, and is thus useful for such measurements.

The PPG/blood oximetry photodetectors can in some embodiments be mounted to view the external ear by reflected light. However in an alternative and often preferred embodiment, the photodetectors may be positioned by the wearable earpiece device to a location inside the ear canal where it can view transmitted light transmitted by the wearable earpieces' light source illuminating the exterior portion of the ear, through the vascular, skin, and cartilage of the ear, and pick up the transmitted light in the ear canal region.

In this embodiment, the system can be configured for continual monitoring of blood oxygen saturation (SPO₂) from the concha area of the ear, and the same or other wearable earpiece mounted infrared sensors, positioned in the ear canal, can also determine the user's body core temperature as well.

Examples of the associated software used to analyze and report the user's temperature are shown in FIG. 4AB and elsewhere.

Body core temperature is also a crucial vital sign used by physicians and parents alike. While sublingual or skin temperature readings have some validity, inner ear temperature readings are accepted as being more representative of body core temperature.

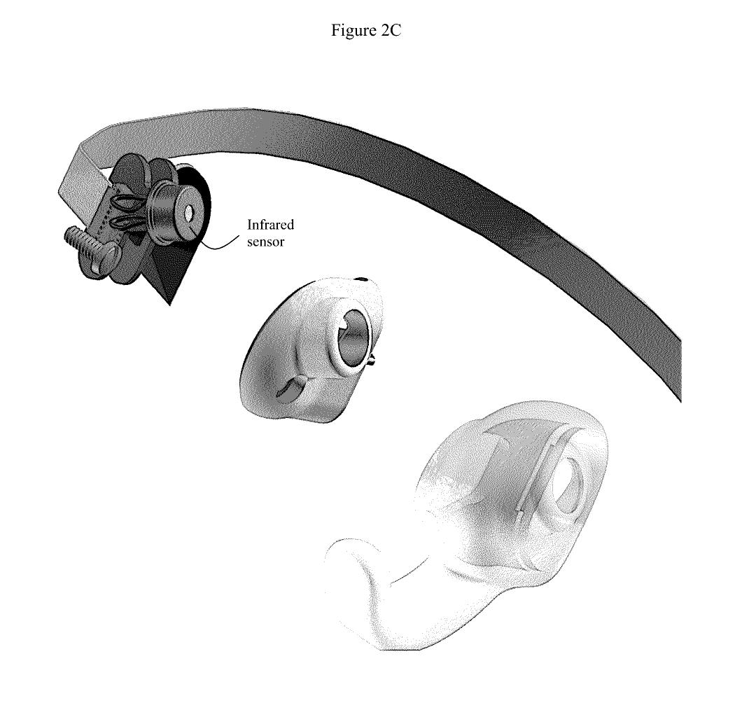

An exploded view of the wearable earpiece is shown in FIGS. 2A and 2B, and a close up of this exploded view is shown in FIG. 2C. As previously discussed, in some embodiments, and as is shown in FIG. 2C, the wearable earpiece (110) will also comprise an infrared detector, such as an infrared (IR) thermopile or other infrared sensor, configured and directed to take readings from the user's inner ear as well. By placing the thermopile just inside the ear canal, the wearable earpiece (110) can ensure an accurate reading can take place, while not impairing airflow or sound into the inner ear to reduce discomfort.

Note that the position and orientation, and light wavelength response characteristics of the infrared sensors needed to measure the use's core body temperature need not be the same as the position and orientation of the optical and infrared sensors used to obtain the previously discussed blood oximetry or PPG measurements. Thus in some embodiments, different sets of infrared sensors may be used for these respective measurements.

To further improve comfort over extended periods, in a preferred embodiment, bulky hardware items such as batteries may been relocated away from the ear towards other regions of the body, where the hardware weight and volume can be more easily borne. Here the previously discussed cable (108) arrangement to the neck worn device (100), or other arrangement, may be used.

Thus to summarize, in some embodiments, the wearable earpiece (110) may additionally or alternatively comprise one or more of either photoplethysmography (PPG) sensors (such as pulse oximeter sensors) configured to obtain user blood oxygen data from the concha region or antitragus region(s) of the user's external ear anatomy; and/or a body core sensor (such as an infrared sensor) configured to obtain user body core temperature measurements from the user's ear canal.

Here, for example, the wearable earpiece can be configured with at least one PPG sensor in the concha, region of the external ear, such as behind the ear's anti-tragus. This sensor can contain optical and infrared LEDs configured to transmit optical and infrared signals into the user's ear. These can be used, in conjunction with various wearable earpiece mounted photodetectors, to determine pulse rates and blood oxygen levels by reflection to one or more wearable earpiece photodetectors.

Alternatively, the wearable earpiece mounted PPG sensor based light sources can transmit optical or infrared signals through the user's ear to wearable earpiece photodetectors, such as infra-red (IR) thermopile detectors, mounted in the user's ear canal, to obtain blood oxygen levels, pulse levels, and the like by optical transmission methods. The wearable earpiece infra-red thermophile detectors can also be used to obtain user body core temperature measurements by infrared emission measurements as well.

Indeed, in some embodiments, the requirement for the user of ECG sensors may be reduced or removed, and the system and method may use only a wearable earpiece (110), either with a self-contained battery, or alternatively deriving power and communications from an external device, such as a Smartphone, through a cable connection.

Examples of how software residing on the external computerized device, such as the previously described smartphone, may be used to initially set up the neck worn device and the wearable earpiece are shown in FIGS. 4A-4B.

### Inputting a User's Health Profile

Note that typically, the system software will also prompt the user to enter in an initial health profile. This health profile will typically then be used by various system algorithms (see the FIG. 3 series) to analyze the sensor data and often make tentative assessments of the user's body status. This health profile input process is shown in FIG. 4C and elsewhere.

Use with Additional Battery Powered Combination Otoscope, Skin Lesion Monitor, and Spirometer

As previously discussed, although in a preferred embodiment, the system and method will use at least three patient worn sensors, use of additional sensors is also contemplated. Not all of these additional sensors need be patient worn. In some embodiments, a separate handheld battery powered “scanning wand” device may be configured with a small video camera, light source, processor, battery, and wireless transceiver may also be used to provide additional medical data such as information pertaining to ear infections (e.g. function as an otoscope device), and/or to function as a skin lesion monitor.

See FIG. 4K for a discussion of how the external computerized device software can be used to manage this scanning wand. When used as a handheld otoscope, the scanning wand will allow a user to hold the otoscope device up to their ear and insert the top of the otoscope inside the user's ear. The otoscope will produce images of the interior of the user's ear, and will use its wireless transceiver to transmit images of the ear to a local computerized device, such as a smartphone, for subsequent analysis, as will be described shortly. The same camera may be used to produce close up pictures of suspicious moles, possible skin cancers, and other skin lesions of potential concern to the user or their healthcare provider.

In other embodiments, this battery power “scanning wand” may additionally operate as a handheld battery powered spirometer. To do this, the scanning wand will typically additionally also comprise an electronic air flow sensor, configured to operate with the previously discussed scanning wand processor, battery, and wireless transceiver. This Spirometer will take air flow readings as the user inhales and exhales, and transmit the air flow data to a local computerized device, such as a smartphone, for subsequent analysis, as will be described shortly.

Use with a Wireless Wrist-Mounted Blood Pressure Cuff

In some embodiments, the system can also be used with a wireless, rechargeable, wrist mounted blood pressure cuff. This is shown in FIG. 5. Data showing how the software can manage this cuff, and integrate cuff blood pressure sensor measurements in with the other sensor measurements for various types of medical diagnoses, is shown in the FIG. 3 series, and also in FIGS. 4E, 4W, 4X and elsewhere.

Neck Mounted Devices with Airflow Sensors

In some embodiments, the system and method (e.g. a neck mounted embodiment) may further contain motion sensors and accelerometers configured to measure the motion of the user's chest as it rises and falls during respiration. With appropriate calibration, such sensors may be configured as an alternative type of spirometer. Examples of how such data may be displayed are shown in FIG. 4Y and elsewhere.

### Software Based Medical Assessment Methods.

One important goal of the invention is to deskill medicine, and help ensure that untrained users can participate in their diagnosis. This approach of diagnosis without trained professional assisting a user, has a significant potential to help those in rural areas, as well as third world and developing countries where personal, facilities, and medical equipment can be limited.

In some embodiments, particularly useful when the system is configured to wirelessly transmit sensor data to local computerized devices such as smartphones, various automatic and user assisted software algorithms and methods may be used to interpret the vital sign data. Examples of these algorithms are shown in the FIG. 3 and FIG. 4 series.

As will be discussed, such methods, even when used by users with limited training or medical knowledge, can quite useful. In the following embodiment, a comparative, visual analysis of a user's medical condition against standardized, known positive and negative images is taught.

### Vital Sign Signal Analysis

Examples of various algorithms and user inputs that may be used for vital sign signal analysis are shown in FIGS. 3A to 3W, as well as FIGS. 4A to 4AD.

In some embodiments, the system may use modified pulse transit time (PTT) algorithms to obtain blood pressure measurements. These modified PTT algorithms were inspired by the work of McCarthy et. al., “An Investigation of Pulse Transit Time as a Non-Invasive Blood Pressure Measurement Method”, Journal of Physics: Conference Series 307 (2011) 012060 and Zheng et. al., “An Armband Wearable Device for Overnight and Cuff-Less Blood Pressure Measurement”, IEEE TRANSACTIONS ON BIOMEDICAL ENGINEERING, VOL. 61, NO. 7, JULY 2014. These methods can use input from the blood pressure monitoring cuff, or other sensors such as the earpiece mounted blood oximeter.

In some embodiments, the algorithms described in the above two papers may be further modified by, for example, either 1) weighting the calculation with the heart rate; and/or 2) using the PTT calculated from R wave of the ECG to the maximum of the PPG; and/or by using R wave and the maximum of the upslope of the PPG.

In the above two research papers, the systolic and diastolic pressure were computed by initially calibrating the PTT with an initial systolic and diastolic blood pressure reading, followed by using a physiological model to estimate the systolic and diastolic pressure based on the changes in the PTT. By contrast, in some embodiments, this method may be modified by instead making use of the user's heart rate stored in memory from the time of the initial calibration, and comparing this with the user's heart rate at the time the current measurement is taken. Using this later approach and the user's heart rate at the time of the present measurement (current time), then the user's systolic pressure is:

User's systolic pressure=(User's systolic pressure computed as per the above references)*(user's heart rate at calibration)/(user's heart rate at the current time)

User's diastolic pressure=(User's diastolic pressure computed as per the above references)*(user's heart rate at calibration)/(user's heart rate at the current time). Examples of blood pressure output data and the user interface are shown in FIG. 4AA and elsewhere.

### Automated or Semi-Automated Diagnosis of Acute Otitis Media

In this embodiment, interpretation of data obtained from the scanning wand when used as a handheld otoscope is taught. This method uses a comparative approach to help diagnose acute otitis media (AOM, also commonly known as an “ear infection”).

In this embodiment, the user uses scanning wand/handheld otoscope to take one or more pictures of their tympanum. The handheld otoscope obtains images, which will typically be transmitted via a wireless transceiver (or alternatively by a direct cable connection) to a computerized device. Here we will use local computerized devices such as smartphones, tablets, and laptops as an example, but of course the data may be transmitted over a network to remote computerized devices run by medical professionals as well, as needed.

Using a smartphone as a preferred embodiment, the otoscope images may be transmitted to the smartphone using wireless (e.g. WiFi, Bluetooth, or other connection), where the images can then be manipulated and interpreted using smartphone (e.g. an app) or other computerized device diagnosis software, which typically interacts with the user via vital sign analysis and interpretation user interface (UI).

Among other functions, this vital sign analysis and interpretation (VSAI) UI can then be used to control the otoscope (and other sensors as well as needed) and for example receive and display images of the users ear drum, optionally on a real-time basis as the images are generated by the handheld otoscope.

The software VSAI UI can also, for example, prompt the user to obtain vital sign sensor data such as by prompting for otoscope video data (e.g. snapshot of the user's eardrum). The software and UI can also be used to help control the focus, adjustment, and lighting of the handheld otoscope as well.

Alternatively or additionally, the software can incorporate at least a certain amount of computer vision or camera control functionality, and can be set to automatically acquire images when the system determines that the otoscope camera is acquiring an adequate image. Additionally or alternatively, the software and UI can provide one or more sample images to help provide a guide for the user, and help insure that the otoscope camera images are of sufficient quality.

Once the otoscope camera has acquired one or more adequate images, which may be displayed on the computerized device screen, in some embodiments the system can then provide a library of various comparative or reference images and put images from this library on screen as well. These comparative images, could, for example, include images of ear drum that are positively and negatively associated with AOM. The user could then be prompted to swipe through the images and selected the images which look the most similar to their own image above. Alternatively the system may use various types of computer vision criteria to make an automated diagnosis. The system may be further set to transfer potentially problematic sets of data to more distant medical professionals for further analysis.

Typically, based on either user selected images, computer selected images, or both, the system software can then use one or more algorithms to process those human or computer selected images and make a tentative diagnosis. In one embodiment, this algorithm may use a weighting system wherein images that are positively identified with a disease process, such as AOM, would have a positive value (such as 1), while images that are negatively correlated with AOM would have a negative value (such as −1). After the user or the software reviews all the images, the software can then process the various selected images to arrive at a sum which could be compared to a threshold number (e.g. 5). Any values above this threshold would be considered positive for AOM, while any values below would be negatively associated for AOM. See FIGS. 4K and 4L and elsewhere to see more specific examples.

Analysis of Spirometer Data

In embodiments where the handheld wand is used as a spirometer, the data may be analyzed according to the examples shown in FIGS. 4M and 4N, and elsewhere.

Obstructive Sleep Apnea Diagnosis

To definitely diagnose obstructive sleep apnea (e.g. sleep apnea with an Apnea Hypopnea Index (AHI)>15, i.e. a cut off that currently defines those with moderate-severe sleep apnea), and use that cut off as a criterion standard for sleep apnea, the system may use a validated metric, here called the “oxygen desaturation index”, or ODI. The ODI here is defined as the hourly average number of oxygen desaturation episodes (as measured by the pulse odometer), which are defined as at least a 4% decrease in oxygen saturation from the average saturation in the preceding 120 seconds, and lasting >10 seconds.

This ODI value can be derived by measuring the total number of desaturations divided by either the total sleep time, or alternatively an arbitrary interval of that time (for example between 1 AM and 6 AM). The limitation in the latter approach is that the user (tester) may not be sleeping during that time. An alternative approach is for the tester to be prompted to enter the sleep start time and sleep end time. The system can be configured to allow either approach.

In one embodiment, the software running on the computerized device can first receive input from the user using an obstructive sleep apnea screening questionnaire (sometimes called a “Stop Bang” questionnaire, see Chung et. al., “Predictive performance of the STOP-Bang score for identfying obstructive sleep apnea in obese patients”, Obes Surg 2013, Dec. 23(12), 2050-7, as well as Chung et al. Anesthesia Analgesia 2012; 114:993-1000, and Ling et al. SLEEP 2012; 35(1):89-96) that has questions that probe if the user has a possible tendency towards sleep apnea. The questionnaire results can then be used by the software in its automated sleep apnea algorithm discussed below.

Automated sleep apnea algorithm: In one embodiment, software running on the computerized device can receive blood oxygen levels from the earpiece device, and determine that if a user has an ODI-4%>15/hr., the sleep apnea diagnosis should be reported as POSITIVE regardless of any feedback from the obstructive sleep apnea screening questionnaire.

Similarly, if the software determines that the user has an ODI-4% of 6-14, then this user may, or may not, have moderate to severe sleep apnea. In this scenario, absent questionnaire results, the results might be considered INDETERMINATE, but if the “STOP-Bang” questionnaire shows that the user is at high risk for moderate-severe OSA, then the results should be reported as POSITIVE. However if the software determines that the ODI-4% is less than or equal to 5, then the results should be reported as NEGATIVE regardless of the STOP-Bang questionnaire results.

See FIG. 3Q for further examples such obstructive sleep apnea software analysis methods. Thus in some embodiments, the system may conduct a plurality of queries comprising any of stop-bang sleep apnea queries, chronic obstructive pulmonary disease queries, hypertension queries, atrial fibrillation queries, and infectious disease queries.

Further, in some embodiments, the health status information collected by the device may be used to determine one or more time durations to control the vital sign monitoring process.

Although shorter times are contemplated, this time duration may often be of one hour duration or greater, and may even be of several days (e.g. 72 hours) duration or greater.

The health status information may be further used to select at least one algorithm comprising any of pulse transit time (PTT) algorithms, ECG modified pulse transit time algorithms, pulse oximeter modified pulse transit time algorithms, or pulse oximeter based sleep apnea diagnosis algorithms.

Note, however, that in some alternative embodiments, a simplified form of the invention's system and method may also be used. In these simplified alternative embodiments, the invention may comprise an earpiece system and method to diagnose obstructive sleep apnea. Here the system and method may comprise an ear wearable pulse oximeter that is configured to work with an external computerized device, such as a smartphone, to receive health status information pertaining to potential sleep apnea issues, and to analyze pulse oximeter date from the ear worn device.

In this simplified embodiment, the invention may comprise an ambulatory patient wearable vital sign monitoring system, comprising at least one ambulatory patient wearable sensor comprising at least one ear wearable pulse oximeter. The ear wearable sensor may be further configured to transmit sensor data to an ambulatory patient wearable computerized device. This ambulatory patient wearable computerized device, which may be a smartphone, may comprise at least one processor, and at least one user interface (such as a touch-screen user interface). This ear wearable ambulatory patient wearable computerized device will typically be configured to use the user interface to query an ambulatory patient with a plurality of queries regarding the ambulatory patient's health history, and receive health status information pertaining to this health history from the ambulatory patient.

This ear worn ambulatory patient wearable computerized device can be further configured to use said this health status information to a) select a time duration and sensor data acquisition parameters of said vital sign monitoring system; and/or select at least one algorithm (such as a sleep apnea algorithm) that uses the health status information and said sensor data to return diagnostic information (e.g. a sleep apnea diagnostic information) pertaining to the health history to ambulatory patient and or other interested parties, either local or remote (e.g. remote internet servers).

Other earpiece mounted sensors, (e.g. accelerometers to detect breathing patterns, microphones to pick up breathing/snoring sounds, and the like), or computerized device sensors (e.g. smartphone based microphones) may also be employed.

### Other Embodiments that Integrate Information from External Whole-Blood In-Vitro Diagnostic Assays

In other embodiments, the system may also be used in conjunction with various types of in-vitro diagnostics, such with an in-vitro assay cartridge based system that uses an integrated lancing device to obtain droplets of either whole blood or urine from the user, blood or urine collection, activator (assay chemistry) storage, and sample dispensing. This in-vitro assay can analyze for medically important analytes such as cholesterol (see FIG. 4R), glucose, hemoglobin (see FIG. 4S), white blood cell levels, hepatitis A, mononucleosis (see FIG. 4T) and common blood or urine chemistry analytes. The system can also integrate these results with information from the patient history, and other sensors, and produce tentative diagnosis as is shown by various examples from the FIG. 3 and FIG. 4 series.

The in-vitro diagnostic component of the invention is generally designed to be used by users who are not medical professionals, and to require less than 20 microliters (less than a drop) of blood or urine per test. The in-vitro diagnostic system then automatically dispenses the drop of sample onto the appropriate reaction zone of a disposable reagent test cartridge that contains the reaction chemistry of interest. This disposable reagent (or test) cartridge also contains a removable cap configured to acquire the test sample. This disposable reagent cartridge is then read by an appropriate electronic in-vitro diagnostic meter.

This electronic in-vitro diagnostic meter can also be used to integrate other components of the invention as well. For example, as is shown in FIG. 5, the meter (diagnostic reader) can also be used as a recharging station to recharge some of the multiple, hot-swappable batteries for the neck device. Additionally, the in-vitro diagnostic reader can be used as a dock/recharging station for the scanning wand (e.g. battery powered otoscope/skin-lesion monitor/spirometer device), or blood pressure cuff. Additionally, the in-vitro diagnostic reader can also be used as a docking/recharging system for a smartphone or other local computerized device.

The in-vitro diagnostic meter may itself often comprise a processor, one or more chambers to hold the disposable reagent cartridges, and various colorimetric, fluorescent, or electrochemical sensors configured to assess the status of the disposable reagent cartridges as the various diagnostic reagents progress. The in-vitro diagnostic meter will also typically be configured to report these reaction results, typically using a wireless transceiver, to various local computerized devices such as smartphones. There the results may analyzed by suitable software, and viewed in context with results obtained from various other sensors from the neck mounted device, wearable earpiece, scanning wand device, and the like.

In-vitro diagnostic assays configured to operate with a droplet of whole blood typically obtain the droplet with the aid of a lancing device (lancet), which is often a spring operated needle held into the proper position by various plastic holders and supports. Here, most prior art lancing devices are pen shaped lancets devices that rely on a spring release trigger mechanism. The user typically holds the lancet in one hand, applies the tip of the lancet to a finger of the other hand, and activates the trigger. The spring then drives the needle tip of the lancet into the finger, producing a small drop of blood. The problem with such prior art lancet devices is that a poor finger stick can result if the user moves their finger during the lancing process, flinches, or otherwise uses poor technique.

To avoid these problems, in some embodiments, the disposable reagent cartridge used for the invention's in-vitro diagnostic device may be configured to integrate a lancing device into a removable cartridge cap. This cartridge cap can then be used to prick the user's finger to obtain the blood sample needed for the assay. This lancing device will typically be configured for one-time use, and will also be configured to only fire when pressed onto the user's finger.

The disposable reagent cartridge cap will typically also be configured with a small capillary tube, usually located directly above the cap's built in lancing device. This small capillary tube is used to draw in the blood (or urine) sample through capillary action. Typically this capillary tube is located very close to the lance portion of the cap, so that the user after lancing their finger to obtain the sample, need only to move their finger a few millimeters to dispense the blood sample into the capillary tube.

The advantage of integrating a small capillary tube into the disposable reagent cartridge cap is that the capillary tube helps insure that the proper and precise amount of sample is then subsequently applied to the reaction chemistry. Here, the amount of sample that is ultimately applied to the reaction chemistry can be controlled by selecting the inner diameter and length of the capillary tube

For urine based assays, the lancing step is not needed. Here the user can simply place the cap with capillary tube into an open vessel containing a urine sample, and the capillary tube will again wick up the required volume of urine by capillary action.

At this point, the user would insert the cap into the larger disposable reagent cartridge. This installation positions the capillary tube over the cartridges assay chemistry (often in the form of a dry reagent test strip absorbent material, but in some embodiments, liquid reagents may also be used). In order to precisely apply the sample, in some embodiments the sample may be dispensed onto the dry reagent test strip absorbent material through an axial or rotary plunger. This plunger can be operated manually or automated within a diagnostic point of care machine. In other embodiments, however, mere contact of the capillary tube with the dry reagent test strip absorbent material will be enough to adequately dispense the simple onto the test strip.

In cases where the assay reaction chemistry uses some liquid components, the plunger can accept a volume of this liquid solution, often contained in a chamber of the reaction cartridge, sealed by a thin foil lid or other thin membrane barrier. As before, when the plunger operates to dispense the blood or urine sample, the plunger moves linearly inside the capillary tube, and thus begins to displace the blood out of the capillary tube and onto the disposable reagent cartridge's absorbent reaction material. However as the plunger continues to move, the foil lid will also eventually become pierced by spikes associated with the plunger. The liquid reaction chemistry (e.g. activator solution) can then flow either onto the absorbent reaction material or (depending on the configuration of the cartridge, cap, fluid chamber, and thin foil lid) will flow into the capillary tube as the plunger continues to be compressed. The net result is that both the reaction chemistry (e.g. the activator solution) and the blood sample can then be precisely deposited on the absorbent material of the disposable reagent cartridge. The assay can then proceed, and the results assessed by changes in various colorimetric, fluorescent, or electrochemical status of the absorbent reaction material as the in-vitro diagnostic test chemical reaction proceeds.

Alternatively the system may be configured to obtain information from electrochemical sensors, such as electrochemical blood glucose monitors, implanted glucose sensing electrodes, and the like. This information may be reported back to the device's processor via wireless links (e.g. Bluetooth, WiFi) or other methods.

Thus in some embodiments, the system may further comprise a plurality of ambulatory patient wearable sensors. These patient wearable sensors can comprise, for example, wearable patient temperature sensors, blood glucose monitors, and patient mounted breathing sensors.

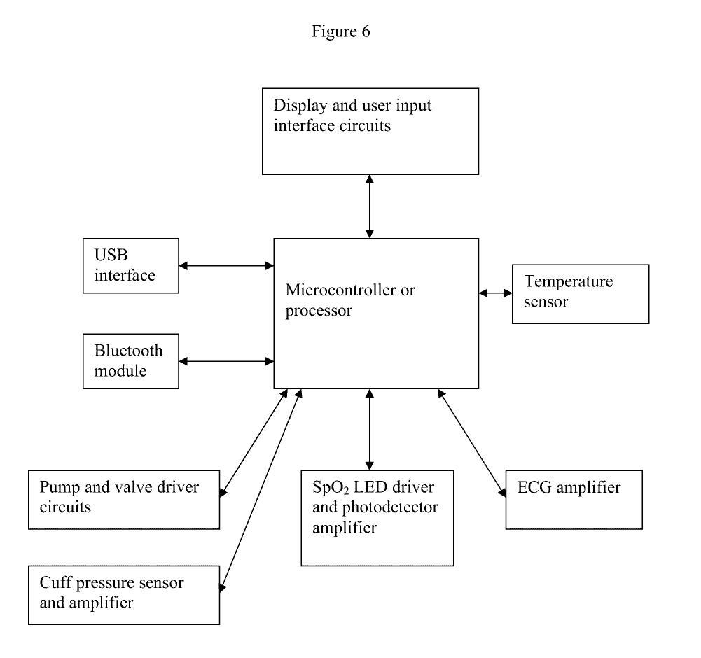

FIG. 6 shows a simplified electrical schematic of a multi-parameter monitoring device produced according to the teaching of parent application Ser. No. 14/186,151, the entire contents of which are incorporated herein by reference.

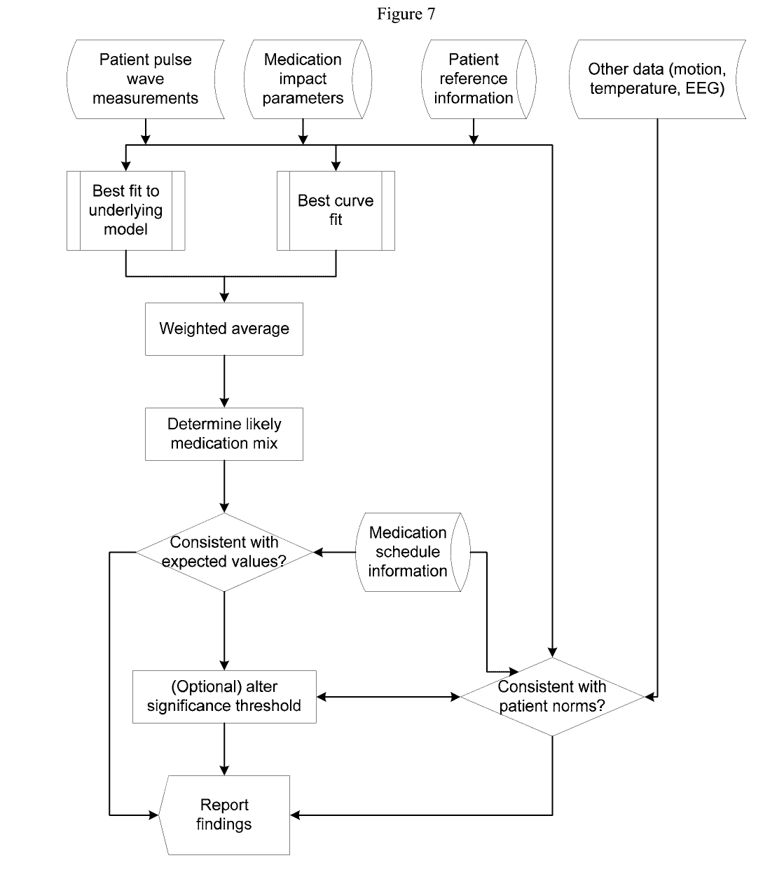

FIG. 7 shows a flow chart showing of some of the various steps that may be carried out the system's processor in order to determine if the various physiological measurements taken by the device's various sensors are showing that the patient is likely following his or her assigned medication schedule, or not, according to the teaching of parent application Ser. No. 15/060,514, the entire contents of which are incorporated herein by reference.

In this embodiment, a variety of different types of patient pulse wave measurements (blood pressure, pulse oximeter, ECG) and other physiological measurements are obtained. This actual data is compared to calculated measurements that would be expected based on the various patient baseline measurements in the absence of medication, schedule of medications, and impact of medications the various patient baseline measurements. If the actual data meets expectations, then the patient is likely adhering to the regime. Depending on which types of data do not meet expectations, non-adherence to various previously described medications may be reported.

Thus in some embodiments, the ambulatory patient wearable computerized device may be configured to (and used) to determine if the patient is following an assigned medication schedule.

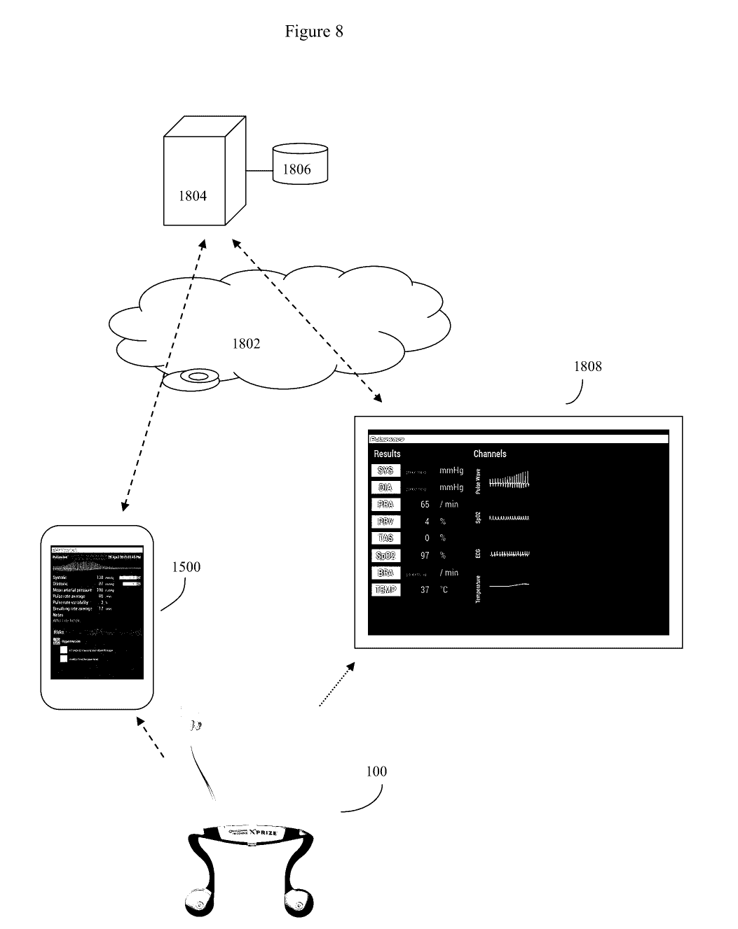

FIG. 8 shows that in some embodiments, the device (100) may also communicate with an external server (1804) and database (1806). For example, the device (100) may send data via a Bluetooth™ or WiFi link to a local Bluetooth equipped smartphone (1500) or WiFi router, and the smartphone or WiFi router in turn may transmit (relay) this data over a network connection (1802) to a remote internet server (1804) and database (1806) for further analysis. The patient or physician can then download this server analyzed data to their local computerized devices (here a tablet device) (1808) for subsequent evaluation, and/or configure the server to automatically react in certain situations.

Thus in some configurations, the ambulatory patient wearable computerized device may further comprise a network interface configured to connect with remote internet servers. Here the ambulatory patient wearable computerized device may be further configured to communicate at least some health status information, sensor data, and/or diagnostic information to these remote internet servers.

1. An ambulatory patient wearable vital sign monitoring system, comprising:
a plurality of ambulatory patient wearable sensors, each wearable sensor comprising at least one ECG sensor, pulse oximeter, and oscillometric blood pressure monitor;
each said wearable sensor further configured to transmit sensor data to an ambulatory patient wearable computerized device;
said ambulatory patient wearable computerized device comprising at least one processor, and at least one user interface;
said ambulatory patient wearable computerized device configured to use said at least one user interface to query an ambulatory patient with a plurality of queries regarding a health history of said ambulatory patient, and receive health status information pertaining to said health history from said ambulatory patient;
said ambulatory patient wearable computerized device further configured to use said health status information to:
a) select a time duration and sensor data acquisition parameters of said vital sign monitoring system; and/or
b) select at least one algorithm that uses said health status information and said sensor data to return diagnostic information pertaining to said health history to at least said ambulatory patient.

2. The system of claim 1, wherein said at least one ECG sensor is a chest wearable ECG sensor, said pulse oximeter is either an ear wearable or a finger wearable pulse oximeter, and said oscillometric blood pressure monitor is a wrist wearable oscillometric blood pressure monitor.

3. The system of claim 1, wherein said at least one ECG sensor comprises a plurality of ECG sensors both attached to, and in electrical communication with, a neck mounted flexible harness;
and wherein both said at least one pulse oximeter and said ambulatory patient wearable computerized device are also both attached to, and in electrical communication with, said neck mounted flexible harness.

4. The system of claim 1, wherein any of said at least one ECG sensor, pulse oximeter, and oscillometric blood pressure monitor are configured to transmit data to said ambulatory patient wearable computerized device using a wireless link.

5. The system of claim 1, wherein said plurality of queries comprise any of stop-bang sleep apnea queries, chronic obstructive pulmonary disease queries, hypertension queries, atrial fibrillation queries, and infectious disease queries.

6. The system of claim 1, wherein said time duration is of one hour duration or greater, and wherein said at least one algorithm comprises any of pulse transit time (PTT) algorithms, ECG modified pulse transit time algorithms, pulse oximeter modified pulse transit time algorithms, or pulse oximeter based sleep apnea diagnosis algorithms.

7. The system of claim 1, wherein said ambulatory patient wearable computerized device comprises a smartphone or tablet device, and wherein said user interface comprises a touch sensitive display screen.

8. The system of claim 1, wherein said ambulatory patient wearable computerized device further comprises a network interface configured to connect with remote internet servers; and
wherein said ambulatory patient wearable computerized device is further configured to communicate at least some health status information, sensor data, and/or diagnostic information to said remote internet servers.

9. The system of claim 1, wherein said plurality of ambulatory patient wearable sensors further comprise any of wearable patient temperature sensors, blood glucose monitors, and patient mounted breathing sensors.

10. The system of claim 1, wherein said ambulatory patient wearable computerized device is further configured to determine if said ambulatory patient is following an assigned medication schedule.

11. A method of obtaining vital signs from an ambulatory patient, said method comprising:
using an ambulatory patient wearable computerized device comprising at least one processor, and at least one user interface to query said ambulatory patient with a plurality of queries regarding a health history of said ambulatory patient, and receive health status information pertaining to said health history from said ambulatory patient;
using said health status information and said ambulatory patient wearable computerized device to select a time duration and sensor data acquisition parameters of a plurality of ambulatory patient wearable sensors, each wearable sensor comprising at least one of an ECG sensor, pulse oximeter, and oscillometric blood pressure monitor;
each said wearable sensor further configured to transmit sensor data to said ambulatory patient wearable computerized device; and
further using said health status information and said ambulatory patient wearable computerized device to select and use at least one algorithm that uses said health status information and said sensor data to return diagnostic information pertaining to said health history to at least said ambulatory patient.

12. The method of claim 11, wherein at said ECG sensor is at least one chest wearable ECG sensor, said pulse oximeter is either an ear wearable or a finger wearable pulse oximeter, and said oscillometric blood pressure monitor is a wrist wearable oscillometric blood pressure monitor.

13. The method of claim 11, wherein said ECG sensor comprises a plurality of ECG sensors both attached to, and in electrical communication with, a neck mounted flexible harness;
and wherein both said at least one pulse oximeter and said ambulatory patient wearable computerized device are also both attached to, and in electrical communication with, said neck mounted flexible harness.

14. The method of claim 11, wherein any of said ECG sensor, pulse oximeter, and oscillometric blood pressure monitor is configured to transmit data to said ambulatory patient wearable computerized device using a wireless link.

15. The method of claim 11, wherein said plurality of queries comprise any of stop-bang sleep apnea queries, chronic obstructive pulmonary disease queries, hypertension queries, atrial fibrillation queries, and infectious disease queries.

16. The method of claim 11, wherein said time duration is of one hour duration or greater, and wherein said at least one algorithm comprises any of pulse transit time (PTT) algorithms, ECG modified pulse transit time algorithms, pulse oximeter modified pulse transit time algorithms, or pulse oximeter based sleep apnea diagnosis algorithms.

17. The method of claim 11, wherein said ambulatory patient wearable computerized device comprises a smartphone or tablet device, and wherein said user interface comprises a touch sensitive display screen.

18. The method of claim 11, wherein said ambulatory patient wearable computerized device further comprises a network interface configured to connect with remote internet servers; and
wherein said ambulatory patient wearable computerized device is further configured to communicate at least some health status information, sensor data, and/or diagnostic information to said remote internet servers.

19. The method of claim 11, wherein said plurality of ambulatory patient wearable sensors further comprise any of wearable patient temperature sensors, blood glucose monitors, and patient mounted breathing sensors.

20. The method of claim 11, wherein said ambulatory patient wearable computerized device is used to determine if said ambulatory patient is following an assigned medication schedule.
